(19) United States
(12) Patent Application Publication
(10) Pub. No.: US 2023/0203090 A1
(43) Pub. Date: Jun. 29, 2023

(54) REAGENTS AND METHODS FOR ANTIBODY SEQUENCING

(71) Applicant: RAPID NOVOR, INC., Kitchener (CA)

(72) Inventors: Thierry LE BIHAN, Guelph, Ontario (CA); Bin MA, Waterloo, Ontario (CA); Paul TAYLOR, Waterloo, Ontario (CA); Chenyu YAO, Waterloo, Ontario (CA); Qixin LIU, Waterloo, Ontario (CA); Chelsea REITZEL, Kitchener, Ontario (CA); Kayle Kathleen Marie GOROSPE, Windsor, Ontario (CA); Mariya LIYASOVA, Kitchener, Ontario (CA)

(21) Appl. No.: 18/009,304

(22) PCT Filed: Jun. 10, 2021
(86) PCT No.: PCT/CA2021/050791

§ 371 Date: Dec. 8, 2022

Related U.S. Application Data
(60) Provisional application No. 63/038,069, filed on Jun. 11, 2020.

(51) Int. Cl. C07K 1/107; C07K 16/42

(52) U.S. Cl. CPC C07K 1/107; C07K 16/42; C12Y 304/21004; C07K 2317/34; C07K 2317/565

(57) ABSTRACT

Methods and reagents to obtaining a sample enriched in peptides comprising the third complementarity-determining region of the heavy chain (CDRH3) of immunoglobulins, such as IgGs, are described. These methods are based on the use of targeted protease digestion of immunoglobulins and affinity purification of CDRH3 peptides using specific antibodies. Such methods and reagents are useful for analyzing the immunoglobulin repertoire.

### CROSS REFERENCE TO RELATED APPLICATIONS

The present application claims the benefit of U.S. provisional patent application No. 63/038,069 filed on Jun. 11, 2020, which is incorporated herein by reference in its entirety.

### TECHNICAL FIELD

The present invention generally relates to the field of immunology, and more specifically to antibody sequencing and assessment of the antibody repertoire.

### BACKGROUND ART

The innate immune system is responsible for the early detection and eradication of pathogens while the adaptive immune system involves the broader and optimized recognition of a repertoire of antigens. In addition of being highly specific, the adaptive immunity also has the characteristic of generating the immunologic memory as part of its arsenal of strategies. Following a first encounter with a pathogen, long-lived memory T and B cells are rapidly established to assist as part of a defense mechanism against this pathogen. A following exposure will trigger activation of memory cells in mounting a robust and specific immune response against pathogens. The T and B cell repertoires undergo precise clonal expansion and antibody affinity enhancement to generate a fine-tuned adaptive immune response including the generation of antibodies.

Antibodies are proteins which are classified as glycoproteins; they are secreted by a specific class of B lymphocytes which are known as plasma cells. They are composed of four polypeptide chains; two identical copies of both heavy (H, 55 kDa) and light (L, 25 kDa) chains held together by disulfide bridge(s). The basic profile is similar to a “Y” shape composed of a crystallizable fragment (Fc) and an antigen binding fragment (F(ab)₂). The F(ab)₂ domain is a dimer composed of a part of the heavy chain (H) and the complete light chain (L), each of them composed of three hypervariable loops named the complementarity determining regions (CDRs). The variable regions are generated by somatic recombination between three gene segments named Variable (V), Diversity (D) and Joining (J). The V(D)J segments are even more diversified upon antigen recognition; the different gene segments are then arranged in a semi-random process. The CDRs are critical for the interaction with antigens. Moreover, the CDR3 from heavy chain (H-chain) CDRH3 is the most diverse of the CDRs and it has been proposed to play a key role in antigen recognition and binding (Xu & Davis, 2000). The CDRs are linked by regions called “framework” which in combination with the CDRs confer the structural support to the F(ab)₂ loops.

Antigen binding properties have made antibodies useful in therapeutics, research and diagnostics, particularly as biomarkers. It is estimated that the total IgG circulating in the blood ranges between 37 g to 60 g. IgG is one of the most abundant proteins found in plasma. However, current research does not show a complete picture of the IgG repertoire, especially their targets, efficiency and distributions. This is mainly due to a lack of tools allowing the direct sequencing of antibodies to strengthen the understanding of the nature and diversity of IgGs.

The most accepted approach to evaluate the IgG repertoire consists of sequencing the B cell population (B Cell repertoire). Each B lymphocyte is characterized by its antigen-specific receptor (BCR). Those repertoires are considerably disturbed during an antigen driven response, particularly in the context of infection or autoimmune syndrome, reflecting an adaptation to the perturbation. Several studies on B and T cell repertoire analysis have been done to evaluate vaccine efficacy (Jackson et al., 2014) and screen for the production of monoclonal antibodies targeting specific antigen(s) (Jardine et al. 2016, Cheung et al., 2012). Exploring the immune characteristics of specific diseases with an emphasis on autoimmune conditions has been recently performed (Bashford-Rogers et al., 2019). Due to constant exposure to different antigens, the B Cell repertoire is continuously changing allowing the evaluation of the relationship between infection, disease and autoimmunity. However, investigation of peripheral B-cells alone does not provide a clear measure of the sequence diversity and richness of polyclonal repertoire in serum.

Immune protection is achieved through the circulating antibodies in serum, not the immunoglobulin receptor on B cells. In addition, it has been observed in a previous study that even if in circulation, some B-cells will not produce any detectable antibody (Chen et al., 2017). Moreover, another study showed that only 2% of the BCR is accessible in circulation at any time (Choudhary & Wesemann, 2018). These studies suggest that the full immune repertoire cannot be truly profiled or characterized through B-cell sequencing. In contrast, directly tackling the pool of IgG proteins using proteomics is a promising approach to assess the diversity and complexity of the immune response. An IgG targeted proteomics approach has been attempted a few times. It often consists of combining deep proteomics sequence coverage with genomics/transcriptomics information (Cheung et al., 2012, Georgiou et al., 2014, Wine et al., 2013, Lavinder 2012). There are two main challenges with the latter approach:

1) Peptide sequence identification mostly depends on database generated from genomics and/or transcriptomics data, which, as described earlier, may not reflect the direct IgG immune repertoire, and

2) There is a major dilution of the peptides from the hypervariable regions in contrast with the more abundant peptides from conserved regions.

A possible way to address challenge 1 would consist of using a complete de novo approach to polyclonal antibody sequencing. Such an approach was performed by Guthal et al., although they reported their polyclonal antibody mixture more closely resembled that of an oligoclonal sample with only a few different mAbs (Guthals et al. 2016). Such samples are much simpler than a complex IgG repertoire.

With regards to challenge 2, the more diversified the antibody pool is, the less detectable that unique CDRs will be as they will be less abundant than the conserved regions. Consequently, due to the CDRH3 region's extreme variability and properties, CDRH3 peptides are often hard to detect. As a result, there is little protein sequencing and proteomic information about CDRH3. Another recently proposed proteomics approach is the nanoSurface and molecular orientation limited (Nsmol) proteolysis technology developed by Shimadzu (Iwamoto et al., 2018; Shumada & Iwamoto 2015). Nsmol relies on the use of a single protease, trypsin, which limits peptide detection.

There is thus a need for the development of novel reagents and methods for antibody sequencing to assess the B-cell repertoire.

The present description refers to a number of documents, the content of which is herein incorporated by reference in their entirety.

### SUMMARY OF THE DISCLOSURE

In a first aspect, the present disclosure provides the following items:

1. A method for obtaining a sample enriched in peptides comprising the third complementarity-determining region of the heavy chain (CDRH3) of an immunoglobulin, the method comprising:
(a) providing an immunoglobulin-comprising sample;
(b) optionally submitting the immunoglobulin-comprising sample to a treatment that modifies lysine residues into residues that are not substrates for lysine endoproteases;
(c) optionally submitting the sample in (a) or (b) to a treatment that modifies cysteine residues into lysine analogue residues or prevents cysteine residues from forming disulfide bonds;
(d) contacting the sample with an endoprotease under conditions suitable for protein digestion to cleave the immunoglobulin into peptides and generate a peptide comprising (i) the CDRH3 and (ii) an epitope comprising the junction (J) region and the first 4 to 25 residues from the constant (C) region of the immunoglobulin;
(e) contacting the peptide-comprising sample in (d) with an anti-CDRH3 peptide ligand, such as an antibody or antigen-binding fragment thereof, that specifically binds to the epitope, thereby forming complexes of the anti-CDRH3 peptide antibody and the CDRH3 peptides present in the sample; and
(f) dissociating the CDRH3 peptides from the complexes, thereby obtaining a sample enriched in peptides comprising CDRH3 of an immunoglobulin.
2. The method of item 1, wherein the treatment of step (c) modification with acrylamide, iodoacetamide or 2-Bromoethylamine hydrobromide.
3. The method of item 1 or 2, wherein the treatment that modifies lysine residues into residues that are not substrates for lysine endoproteases comprises acetylation, dimethylation, guanidization, or carbamylation.
4. The method of any one of items 1 to 3, wherein the immunoglobulin is a human or non-human primate immunoglobulin, or from mammalian species preferably a mouse, sheep, rabbit or human immunoglobulin.
5. The method of any one of items 1 to 4, wherein the immunoglobulin is of the IgG, IgM or IgA class.
6. The method of any one of items 1 to 5, wherein the epitope is located in a region that overlaps the J region and the C region of the heavy chain of the immunoglobulin.
7. The method of item 6, wherein the epitope is of the sequence VTVSSASTK (SEQ ID NO:1).
8. The method of any one of items 1 to 5, wherein the epitope is located in the first 15 residues from the C region of the heavy chain of the immunoglobulin.
9. The method of item 8, wherein the epitope is of the sequence GPSVFPLAP (SEQ ID NO:2), SVFPLA (SEQ ID NO:3) or AST(KMe₂)GPSVFP (SEQ ID NO:4).
10. The method of any one of items 1 to 9, wherein the anti-CDRH3 peptide antibody is a monoclonal or polyclonal antibody.
11. The method of item 10, wherein the anti-CDRH3 peptide antibody is a monoclonal antibody comprising the following combination of complementarity-determining regions (CDRs):
V_{H} CDR1: GFSLSSY (SEQ ID NO:5) or a variant thereof having one mutation;
V_{H} CDR2: DANDY (SEQ ID NO:6) or a variant thereof having one mutation;
V_{H} CDR3: YSRDGAIDPYFKI (SEQ ID NO:7) or a variant thereof having one mutation;
V_{L} CDR1: QSSQSVAGNRWAA (SEQ ID NO:8) or a variant thereof having one mutation;
V_{L} CDR2: QASKVTS (SEQ ID NO:9) or a variant thereof having one mutation; and
V_{L} CDR3: AGGYSGEFWA (SEQ ID NO:10) or a variant thereof having one mutation;
or
V_{H} CDR1: GFSFSSGY (SEQ ID NO:11) or a variant thereof having one mutation;
V_{H} CDR2: DISGPY (SEQ ID NO:12) or a variant thereof having one mutation;
V_{H} CDR3: TDPTISSSYFNL (SEQ ID NO:13) or a variant thereof having one mutation;
V_{L} CDR1: QSSQSVYKNNRLA (SEQ ID NO:14) or a variant thereof having one mutation;
V_{L} CDR2: LASTLAS (SEQ ID NO:15) or a variant thereof having one mutation; and
V_{L} CDR3: QAYYDGYIWA (SEQ ID NO:16) or a variant thereof having one mutation.
12. The method of any one of items 1 to 11, wherein the anti-CDRH3 peptide antibody is bound to a solid support.
13. The method of item 12, wherein the solid support are beads or a monolithic column.
14. The method of item 13, wherein the beads are protein A- or protein G-conjugated beads, preferably protein G-conjugated beads.
15. The method of any one of items 1 to 14, wherein dissociating the CDRH3 peptides from the complexes is performed by acid elution and/or using an organic solvent.
16. The method of any one of items 1 to 14, wherein the endoprotease is trypsin, a trypsin-like endoprotease, Lys-C, Lys-N, Asp-N, Glu-C, Pro/Ala protease, Sap9, KEX2, IdeS or IdeZ, preferably a lysine endoprotease such as trypsin, a trypsin-like endoprotease, Lys-C or Lys-N.
17. The method of any one of items 1 to 16, further comprising contacting the sample with a second protease.
18. The method of item 17, wherein the second protease is pepsin, chymotrypsin, proteinase K, Glu-C or Asp-N.
19. The method of any one of items 1 to 18, further comprising enriching the immunoglobulin-comprising sample in immunoglobulins prior to performing step b, c or d.
20. The method of item 19, wherein enriching the immunoglobulin-comprising sample in immunoglobulins comprises contacting the immunoglobulin-comprising sample with protein A- or protein G-conjugated solid support, preferably protein A- or protein G-conjugated beads.
21. The method of any one of items 1 to 20, further comprising removing or inactivating the endoprotease and, if present, the second protease, prior to performing step (e).
22. The method of any one of items 1 to 21, further comprising removing from the sample the reagents used for endoprotease digestion prior to performing step (e).
23. The method of any one of items 1 to 22, wherein the immunoglobulin-comprising sample is a biological sample or a cell culture sample.
24. The method of item 23, wherein the biological sample is a blood-derived sample, saliva, nasal secretion, bronchoalveolar lavage, cerebrospinal fluid or lymph.
25. The method of item 24, wherein the blood-derived sample is a plasma or serum sample.
26. The method of any one of items 23 to 25, wherein the immunoglobulin-comprising sample is obtained from a naïve subject or a subject from an infection, an autoimmune disease, a cancer (e.g., multiple myeloma) or from a vaccinated subject.
27. The method of item 26, wherein the immunoglobulin-comprising sample is obtained from a subject suffering from plasma cell dyscrasia.
28. The method of any one of items 1 to 27, further comprising analyzing or characterizing the peptides comprising CDRH3 of an immunoglobulin obtained in step (f).
29. The method of item 28, wherein the analyzing or characterizing is performed by mass spectrometry, preferably liquid chromatography-mass spectrometry (LC-MS).
30. The method of item 28 or 29, wherein the analyzing or characterizing comprises determining the amino acid sequence of the CDRH3 of the peptides from the sample obtained in step (f).
31. An anti-CDRH3 peptide antibody or an antigen-binding fragment thereof that specifically binds to an antigen of 5 to 12 amino acids comprising a sequence that (i) overlaps the junction (J) region and the constant (C) region of an immunoglobulin; or (ii) is within the first 15 residues from the C region of an immunoglobulin.
32. The anti-CDRH3 peptide antibody or an antigen-binding fragment thereof of item 31, wherein the immunoglobulin is a human or non-human primate immunoglobulin, preferably a human immunoglobulin.
33. The anti-CDRH3 peptide antibody or an antigen-binding fragment thereof of item 31 or 32, wherein the immunoglobulin is of the IgG class.
34. The anti-CDRH3 peptide antibody or an antigen-binding fragment thereof of any one of items 31-33, wherein the antigen comprises a sequence that overlaps the J region and the C region of the immunoglobulin.
35. The anti-CDRH3 peptide antibody or an antigen-binding fragment thereof of item 34, wherein the sequence is VTVSSASTK.
36. The anti-CDRH3 peptide antibody or an antigen-binding fragment thereof of item 35, wherein the anti-CDRH3 peptide antibody comprises the following combination of complementarity-determining regions (CDRs):
V_{H} CDR1: GFSLSSY (SEQ ID NO:5) or a variant thereof having one mutation;
V_{H} CDR2: DANDY (SEQ ID NO:6) or a variant thereof having one mutation;
V_{H} CDR3: YSRDGAIDPYFKI (SEQ ID NO:7) or a variant thereof having one mutation;
V_{L} CDR1: QSSQSVAGNRWAA (SEQ ID NO:8) or a variant thereof having one mutation;
V_{L} CDR2: QASKVTS (SEQ ID NO:9) or a variant thereof having one mutation; and
V_{L} CDR3: AGGYSGEFWA (SEQ ID NO:10) or a variant thereof having one mutation;
or
V_{H} CDR1: GFSFSSGY (SEQ ID NO:11) or a variant thereof having one mutation;
V_{H} CDR2: DISGPY (SEQ ID NO:12) or a variant thereof having one mutation;
V_{H} CDR3: TDPTISSSYFNL (SEQ ID NO:13) or a variant thereof having one mutation;
V_{L} CDR1: QSSQSVYKNNRLA (SEQ ID NO:14) or a variant thereof having one mutation;
V_{L} CDR2: LASTLAS (SEQ ID NO:15) or a variant thereof having one mutation; and
V_{L} CDR3: QAYYDGYIWA (SEQ ID NO:16) or a variant thereof having one mutation.
37. The anti-CDRH3 peptide antibody or an antigen-binding fragment thereof of any one of items 31-33, wherein the antigen comprises a sequence this is within the first 15 residues from the C region of the immunoglobulin.
38. The anti-CDRH3 peptide antibody or an antigen-binding fragment thereof of item 37, wherein the sequence is GPSVFPLAP.
39. The anti-CDRH3 peptide antibody or an antigen-binding fragment thereof of any one of items 31 to 38, wherein the anti-CDRH3 peptide antibody is a polyclonal antibody.
40. A method for producing the anti-CDRH3 peptide antibody of any one of items 31 to 39, comprising administering the antigen to an animal and isolating the anti-CDRH3 peptide antibody from a biological sample from the animal.
41. The method of item 40, wherein the antigen is conjugated to a vaccine carrier.
42. The method of item 41, wherein the vaccine carrier is a polysaccharide or a polypeptide.
43. The method of any one of items 40 to 42, wherein the antigen is administered in combination with a vaccine adjuvant.
44. The method of any one of items 40 to 43, wherein the animal is a rabbit.

Other objects, advantages and features of the present invention will become more apparent upon reading of the following non-restrictive description of specific embodiments thereof, given by way of example only with reference to the accompanying drawings.

### BRIEF DESCRIPTION OF DRAWINGS

In the appended drawings:

FIG. 1 shows the consensus sequence around the hypervariable region CDRH3 of human IgGs. The CDRH3 is adjacent to the amino acid sequences “CAR” on the N-terminal side (end of the heavy chain variable region, IGHV) and a longer stretch, ending with “SSASTK” on the C-terminal end (start of the heavy chain constant region). Within the sequence, the amino acid lysine is rarely encountered.

FIG. 2 shows the consensus sequence around the hypervariable region CDRH3 across Homo sapiens IgG. The CDRH3 located between the amino acid sequences “CAR” on the N-terminal side and a longer stretch, ending with a cysteine (modified to a lysine analogue) on the C-terminal end in the heavy chain constant region of the antibody.

FIG. 3 shows a schematic representation of the workflow of the overall procedure. Magnetic beads (protein A or G) (1) are used to capture the raised antibody, anti-human CDRH3 (α-hCDRH3) against peptides emCDRH3 (2). An IgG mixture is subjected to derivatization (cysteine residues reduced and modified and lysine can be derivatized as well) followed by trypsin digestion or Lys-C digestion (3). Prior to incubation of the α-hCDRH3 with the peptide mixture, protease inhibition (heat or pH change) or addition of a protease inhibitor is needed to avoid digesting the α-hCDRH3 antibody. After incubation, the enriched peptides bounded to the antibody α-hCDRH3 are washed, eluted and analyzed by LC-MS.

FIG. 4 shows an MS/MS spectrum of a short peptide containing the targeted epitope sequence VTVSSASTK (SEQ ID NO:1). The spectrum was acquired in HCD mode (MS/MS spectra dominated by b and y ions). Very distinctive C-term fragment ions (y ions) are found and are typical of an emCDRH3 peptide. Even if a given CDRH3 peptide cannot be fully sequenced, identifying such specific ions should allow confirmation of the efficacy of the enrichment method (i.e. this confirmation will permit optimization of the method shown in FIG. 3). The conserved C-terminal sequence generates mostly common ion signatures (in this case, y2, y3, y4, y5, y6, y7, y8, y9 and y10 ions) across all enriched emCDRH3 peptides.

FIG. 5 shows a Venn diagram of the number of MS/MS spectra having a hCDRH3 signature based on either a combination of y5,6,7 or y6,7,8 or y7,8,9. Description of Plasma 1 and 2 is detailed in Example 2.

FIG. 6 is a graph showing the number of spectra in a given LC-MS run having the y6, y7, y8 signature ions typical of a peptide having the emCDRH3 sequence. The sample was a tryptic digest of human plasma (from 870 μg total protein), the enrichment was performed using 18 μL of protein G slurry beads (plasma 1). There is a total of 1955 MS/MS spectra having the ion signature y6, y7, y8. Plasma 2 was the same type of digest, same amount of tryptic digest although performed using 50 μL of the protein G beads slurry, with 4470 spectra having the y6,7,8 signature. In a non-enriched sample, 2 μg of the same plasma digest was loaded on the LC column (estimated maximum capacity in term of peptide load on column), only 33 spectra having the y6,7,8 signature were identified.

FIG. 7 shows the peptide coverage of the trypsinized Promega standard antibody (IgG1, CS302902) after enrichment using the batch 1 antibody. Most of the enriched peptides contained the VTVSSASTK sequence.

FIG. 8 depicts an MS/MS spectrum and sequence assignment VSYLSTASSLDYWGQGTLVTVSSASTK (SEQ ID NO:17) as a 3+ at 936.80278 amu. A good coverage of the almost total length of the sequence is observed (from y2 to y17 and from b2 to b14) confirming in that specific case the enriched peptide contains the complete CDRH3 segment.

FIG. 9 depicts MS spectra signal averaged between 32 to 35 minutes for the entire tryptic peptide digest (upper spectrum) and the enrichment CDR3 region (bottom spectrum). A magnification of the MS region around 936.8 amu is shown in the left side of the bottom spectrum.

FIG. 10 shows the peptide coverage of the Promega standard antibody digested with Asp-N after enrichment using the batch 1 antibody (α-hCDRH3 antibody named PD025). Most enriched peptides contained the VTVSSASTK epitope sequence.

FIG. 11 shows MS/MS spectra and sequence assignment DYWGQGTLVTVSSASTKGPSVFPLAPSSKSTSGGTAALGCLVK (SEQ ID NO:18) as a 4+ at 1068.29797amu. A good coverage of the sequence is observed (from y2 to y19 and less from the N-terminal from b2 to b9) confirming in that specific case the enriched peptide contains the complete CDRH3 segment.

FIG. 12 depicts the reconstruction of the C-term tryptic fragments for the different IgG isotypes for Rhesus monkey (left) or crab eating macaque (right). Human and crab-eating macaque samples show better similarity regarding the C-term of the CDRH3 region.

### DETAILED DISCLOSURE

The present description refers to a number of documents, the content of which is herein incorporated by reference in their entirety.

The use of the terms “a” and “an” and “the” and similar referents in the context of describing the invention (especially in the context of the following claims) are to be construed to cover both the singular and the plural, unless otherwise indicated herein or clearly contradicted by context.

The terms “comprising”, “having”, “including”, and “containing” are to be construed as open-ended terms (i.e., meaning “including, but not limited to”) unless otherwise noted.

Recitation of ranges of values herein are merely intended to serve as a shorthand method of referring individually to each separate value falling within the range, unless otherwise indicated herein, and each separate value is incorporated into the specification as if it were individually recited herein. All subsets of values within the ranges are also incorporated into the specification as if they were individually recited herein.

All methods described herein can be performed in any suitable order unless otherwise indicated herein or otherwise clearly contradicted by context.

The use of any and all examples, or exemplary language (“e.g.”, “such as”) provided herein, is intended merely to better illustrate the invention and does not pose a limitation on the scope of the invention unless otherwise claimed.

No language in the specification should be construed as indicating any non-claimed element as essential to the practice of the invention.

Herein, the term “about” has its ordinary meaning. The term “about” is used to indicate that a value includes an inherent variation of error for the device or the method being employed to determine the value, or encompass values close to the recited values, for example within 10% of the recited values (or range of values).

Unless otherwise defined, all technical and scientific terms used herein have the same meaning as commonly understood by one of ordinary skill in the art to which this invention belongs.

The scope of the claims should not be limited by the preferred embodiments set forth in the examples, but should be given the broadest interpretation consistent with the description as a whole.

The present technology is based on the fact that the CDR3 from the heavy chain (H-chain), CDRH3 is flanked by conserved regions, which was exploited to develop enrichment strategies by combining some or all of the following techniques:

(1) amino acid modification,

(2) the usage of specific proteases, and

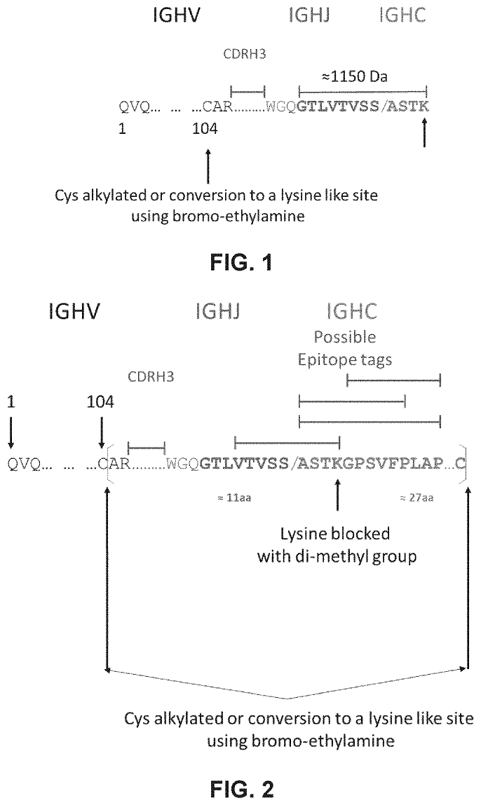

(3) peptide enrichment by immuno-enrichment against a specific sequence. FIG. 1 illustrates an example for human IgG in the vicinity of the CDRH3.

Using human IgG as a representative example, the conserved cysteine residue at position 104 as per the IMGT numbering scheme of the variable region of the heavy chain can be modified in presence of bromo-ethylamine or other suitable reagents to convert this amino acid into a lysine analogue, transforming the sequence into a substrate for lysine endoproteases such as trypsin, trypsin-like proteases, Lys-C or Lys-N. By converting cysteine to a lysine analogue and using the lysine endoprotease Lys-C, the CDRH3 region is then found within a peptide sequence between the last two or three residues amino-terminal to the CDRH3 and a sequence comprising the J region and the first three or four residues of the C region.

If using trypsin only (which cleaves at the C-terminal of lysine and arginine residues), the following peptide is generated: Ex 2: [R]/Nterm-CDRH3-WGQGTLVTVSSASTK-Cterm

These peptides, which are herein referred to as “embedded CDRH3”, “emCDRH3” or simply “CDRH3” peptide, contain the CDRH3 sequence as well as additional amino acids on both extremities. Since lysine residues are rarely encountered within the CDRH3 region (Shi et al. 2014), digestion with a suitable lysine endoprotease such as Lys-C or even trypsin result in a complete emCDRH3 peptide containing this hypervariable region and a conserved sequence tag. An example of a generic emCDRH3 peptide is shown in FIG. 1.

An antibody is then raised against the conserved C-term sequence of the emCDRH3 peptide to perform an enrichment step. The length of the peptide antigen is optimized to confer proper antigenicity and specificity. If too short (for example, the sequence “ASTK” for human), there will be an observed lack of antigenicity; if too long (e.g., the sequence “WGQGTLVTVSSASTK” (SEQ ID NO:19) is quite common within emCDRH3 peptides), the number of false negative can be too important as other sequence variant exist and the enrichment outcome can result in a significant bias in the detection of a sub-population of the emCDRH3 sequences that contain only the “WGQGTLVTVSSASTK” at the expense of the other possible present sequences. For human emCDRH3 enrichment, a rabbit polyclonal antibody may be generated using the antigen peptide “CVTVSSASTK” (SEQ ID NO:20). The cysteine at the N-terminal of the antigen is added to allow peptide attachment to either an antigenic protein carrier (to increase immunogenicity) or to permit antigen coupling to the solid support for the affinity purification of the rabbit polyclonal antibody.

The sequence “VTVSSASTK” (SEQ ID NO:1) is conserved in human IgG and thus antibodies raised against this sequence are useful to enrich for emCDRH3 peptides from human IgG antibodies. However, the method disclosed herein may be adapted for enrichment of emCDRH3 peptides from antibodies from other species using corresponding sequences present in such antibodies. Table 1 below provides the sequences of the C-terminal portion of the emCDRH3 peptides from different species (IgG).

TABLE 1
Different species and the conserved C terminal Lys-C digest of the
emCDRH3 peptide (J/C region).

| | |
|---|---|
| Human: | SSASTK (SEQ ID NO: 21) |
| Rabbit: | SSGQPK (SEQ ID NO: 22) |
| Mouse: | TVSSAK (SEQ ID NO: 23) or TVSAAK (SEQ ID NO: 24) |
| Sheep: | STTPPK (SEQ ID NO: 25) |
| Rhesus (Macaca mulatta) | SSASTK (IgG1) (SEQ ID NO: 21) |
| Alpaca (Vicugna paces) | SSASTK (SEQ ID NO: 21) |
| Macaca fascicularis | SSASTK (IgG1) (SEQ ID NO: 21) |

As can be seen from the table, the sequences usually end with a lysine on the C-terminal end, which can be exploited by the use of specific lysine endoproteases (e.g., trypsin or Lys-C), as described above for human IgG antibodies.

Although the sequence “VTVSSASTK” is relatively conserved in humans, it is common to observe some variant forms. So, in an alternative embodiment, the method of the present disclosure may use a nearby sequence that is more conserved, which could reduce the number of false negatives.

Such alternative method targets a different peptide sequence which is significantly more conserved across both H. sapiens and different species (and different IgG isotypes). However, in order to use this more conserved region for enrichment of emCDRH3 peptides with the specific antibodies, the following steps are involved:
1) Modification of the C-terminal lysine residue (e.g., blocking the lysine with a dimethyl group or carbamylation) to inhibit lysine endoprotease (e.g., trypsin, lys-N or lys-C) digestion at this residue;
2) Modification of the cysteine residues at the C-terminal end of the IGHV and in the IGHC into a lysine analogue (e.g., thiol-ethylamine) cleavable by lysine endoproteases;
3) Digestion with a lysine endoprotease (e.g., Lys-C) to generate longer emCDRH3 peptides; and
4) Enrichment of emCDRH3 peptides using an antibody against the short epitope “SVFPLA” (SEQ ID NO:3), AST(KMe₂)GPSVFP (where Mee stand for dimethylation) (SEQ ID NO:4) or “GPSVFPLAP” (SEQ ID NO:2) present in the IGHC. A cysteine is added at either the N-terminal or C-terminal end for simple peptide chemistry purpose only and is not needed for antigenicity (i.e. coupling antigen to solid support for antibody purification for example).

The longer emCDRH3 peptides have a length between 39aa to 61aa (4.2 kDa to 6.7 kDa) depending on the length of the CDRH3, thus involving mid-down proteomics analysis or a second round of protease digestion to generate shorter peptides following the initial enrichment. An example of such an emCDRH3 peptide is shown in FIG. 2.

The method disclosed herein permits the generation of highly enriched fractions of peptide containing the CDRH3 sequence which are sequence by mass spectrometry. The simple generation of such a large spectral dataset can be used, for example, for training purposes to refine algorithms to predict those sequences. By using the above-described digestion procedure, all emCDRH3 peptides should have specific fragment ion signatures from the C-terminal end with either specific y or z ions (for example y1, y2, y3, y4, y5, y6 . . . ).

The method described herein may be modified as needed. For example, to improve sequence coverage, the emCDRH3 peptides may be modified, for example, using C-terminal and D/E modification with a methyl ester of arginine (i.e. which should increase charge state thus reducing m/z and allow a longer stretch of amino acids to be sequenced). In addition, the peptide mixture can be digested a second time with other proteases, for example with less specific enzymes such as pepsin or chymotrypsin, and/or with more specific enzymes such as Asp-N.

Accordingly, in an aspect, the present disclosure provides a method for obtaining a sample enriched in peptides comprising the third complementarity-determining region of the heavy chain (CDRH3) of an immunoglobulin, the method comprising:
(a) providing an immunoglobulin-comprising sample;
(b) optionally submitting the immunoglobulin-comprising sample to a treatment that modifies lysine residues into residues that are not substrates for lysine endoproteases;
(c) optionally submitting the sample in (a) or (b) to a treatment that modifies cysteine residues, for example into lysine analogue residues such as thiol-ethylamine;
(d) contacting or incubating the sample in (c) with an endoprotease, such as a lysine endoprotease, under conditions suitable for immunoglobulin digestion, thereby cleaving the immunoglobulin into peptides including peptides that comprise the CDRH3 as well as an epitope spanning the junction (J) region and the constant (C) region, or located in the first 5, 10, 15, 20 or 25 residues from the C region, of the immunoglobulin;
(e) contacting the peptide-comprising sample in (d) with an anti-CDRH3 peptide ligand, such as an antibody or an antigen-binding fragment thereof, that specifically binds to the epitope, thereby forming complexes of the anti-CDRH3 peptide ligand (e.g., antibody or antigen-binding fragment thereof) and the CDRH3 peptides present in the sample; and
(f) dissociating the CDRH3 peptides from the complexes, thereby obtaining a sample enriched in peptides comprising CDRH3 of an immunoglobulin.

in another aspect, the present disclosure provides a method for obtaining a sample enriched in peptides comprising the third complementarity-determining region of the heavy chain (CDRH3) of an immunoglobulin, the method comprising:
(a) providing an immunoglobulin-comprising sample;
(b) submitting the immunoglobulin-comprising sample to a treatment that modifies lysine residues into residues that are not substrates for lysine endoproteases;
(c) submitting the sample in (a) or (b) to a treatment that modifies cysteine residues, for example into lysine analogue residues such as thiol-ethylamine;
(d) contacting or incubating the sample in (c) with an endoprotease, such as a lysine endoprotease, under conditions suitable for immunoglobulin digestion, thereby cleaving the immunoglobulin into peptides including peptides that comprise the CDRH3 as well as an epitope spanning the junction (J) region and the constant (C) region, or located in the first 5, 10, 15, 20 or 25 residues, preferably in the first 15 residues, from the C region, of the immunoglobulin;
(e) contacting the peptide-comprising sample in (d) with an anti-CDRH3 peptide ligand, such as an antibody or an antigen-binding fragment thereof, that specifically binds to the epitope, thereby forming complexes of the anti-CDRH3 peptide ligand (e.g., antibody or antigen-binding fragment thereof) and the CDRH3 peptides present in the sample; and
(f) dissociating the CDRH3 peptides from the complexes, thereby obtaining a sample enriched in peptides comprising CDRH3 of an immunoglobulin.

Certain aspects of the embodiments concern obtaining an immunoglobulin-comprising sample from a subject. Immunoglobulin-comprising samples can be directly taken from a subject or can be obtained from a third party. Immunoglobulin-comprising samples include, but are not limited to blood-derived samples (e.g., blood, serum, plasma), mucosa (e.g., saliva), lymph, urine, milk, genitourinary secretions, nasal secretion, bronchoalveolar lavage, cerebrospinal fluid, and solid tissue samples (e.g., lymph nodes, tumors). In some aspects, the immunoglobulins may be isolated from a sample comprising B cells, such as B cells from bone marrow, spleen, lymph node, peripheral blood or a lymphoid organ. The sample may be obtained from a normal healthy subject or from a subject/patient suffering from a disease or a condition, including a tumor (e.g., myeloma), an infectious disease, or an autoimmune disease, or from a subject who has been immunized. The sample may be a biological sample obtained from any animal, including non-human primates or humans. In an embodiment, the immunoglobulin-comprising sample is a biological sample from a human. The sample may alternatively be a cell culture sample, for example a cell culture sample comprising hybridomas.

In an embodiment, the method comprises isolating or enriching the immunoglobulins in the sample. In another embodiment, the method comprises isolating or enriching one or more selected classes of immunoglobulins, such as IgG, IgM, IgA, IgE, and/or other major Ig classes. Such methods may include contacting a sample comprising the immunoglobulins with an agent that binds to immunoglobulins or to a specific immunoglobulin class such as protein L (that binds to representatives of all antibody classes, including IgG, IgM, IgA, IgE and IgD), antibodies specific from a given class (e.g., anti-IgG, anti-IgA or anti-IgM antibodies), or proteins such as protein A or protein G that can bind certain immunoglobulin classes, notably IgG.

In an embodiment, the method does not comprise optional step (b). In another embodiment, the method comprises optional step (b). Step (b) comprises treating the sample with suitable reagents to modify the lysine residues, and more particularly the side chain of the lysine residues, in such a way that they are no longer substrates for lysine endoproteases. Methods to modify lysine residues are known in the art, and include, for example acetylation, methylation (e.g., dimethylation), guanidization, or carbamylation. In an embodiment, step (b) comprises treating the sample to add one or more methyl groups on the lysine residue(s), preferably two methyl residues (dimethylation).

In an embodiment, the method does not comprise optional step (c). In another embodiment, the method comprises optional step (c). Methods to modify cysteine residues into lysine analogue residues are known in the art. For example, the cysteine residues may be reduced followed by modification with reagents such as a 2-haloethylamine compound (e.g., 2-bromoethylamine hydrobromide) that adds a group that is similar to the lysine side chain to the residue (e.g., a thiol ethylamine group). Such modified residues, i.e. lysine or lysine analogue residues, are recognized by lysine endoproteases that cleave proteins/peptides in the vicinity of the lysine or lysine analogue residues, e.g. at the N- or C-terminal of the residue. Any suitable lysine endoprotease may be used in the methods described herein. The lysine endoprotease may specifically cleave at lysine residues such as Lys-C or Lys-N, or may cleave at lysine residues as well as other residues such trypsin/trypsin-like proteases that also cleaves at arginine residues. In an embodiment, the lysine endoprotease is an enzyme that specifically cleaves at lysine residues, for example Lys-C or Lys-N. In another embodiment, the lysine endoprotease is an enzyme that cleaves at lysine residues as well as other residues, for example trypsin. A mixture of lysine endoproteases (e.g. Trypsin/Lys-C mix) may also be used. In an embodiment, cysteine residues may be modified to prevent formation of disulfide bonds, for example using acrylamide or iodoacetamide.

In an embodiment, the methods disclosed herein encompass the use of any proteases generating peptide fragments that will contain the entire or fragment of the CDRH3 and the targeted epitope which could include lysine endoprotease (trypsin, Lys-C, Lys-N) but as well protease used for middle down proteomics such as Asp-N, Glu-C, Pro/Ala protease, Sap9, KEX2, IdeS and IdeZ to name a few.

The sample that has been treated with the endoprotease such as lysine endoprotease to generate digestion peptides (i.e. peptide-comprising sample) is then contacted with an anti-CDRH3 peptide antibody or an antigen-binding fragment thereof for enrichment of the CDRH3 peptides. In an embodiment, the method further comprises inactivating or removing the endoprotease(s) present in the sample prior to performing the next step which involves contacting the sample with the anti-CDRH3 peptide antibody or antigen-binding fragment thereof to avoid or minimize the digestion of the anti-CDRH3 peptide antibody or antigen-binding fragment thereof. Inactivation of the endoprotease(s) may be achieved using any method known in the art, for example using one or more suitable protease inhibitors. The method may also comprise inhibiting (using protease inhibitor) and/or removing from the sample the reagents used for endoprotease digestion (using solid phase extraction, SPE, for example) prior to contacting the sample with the anti-CDRH3 peptide antibody or antigen-binding fragment thereof.

The term “anti-CDRH3 peptide ligand” as used herein refers to any molecule that is able to specifically bind to a specific epitope in the CDRH3 peptide, such as a peptide, antibody, antibody fragment, antibody-like molecule, aptamer (nucleic acid or peptide aptamer), etc. The term “antibody or antigen-binding fragment thereof” as used herein refers to any type of antibody/antibody fragment including monoclonal antibodies (including full-length monoclonal antibodies), polyclonal antibodies, multispecific antibodies, humanized antibodies, CDR-grafted antibodies, chimeric antibodies and antibody fragments so long as they exhibit the desired antigenic specificity/binding activity (ability to bind to a specific epitope in the CDRH3 peptide). Antibody fragments comprise a portion of a full-length antibody, generally an antigen binding or variable region thereof. Examples of antibody fragments include Fab, Fab′, F(ab′)₂, and Fv fragments, diabodies, linear antibodies, single-chain antibody molecules (e.g., single-chain Fv, scFv), single domain antibodies (e.g., from camelids), shark NAR single domain antibodies, and multispecific antibodies formed from antibody fragments. Antibody fragments can also refer to binding moieties comprising CDRs or antigen binding domains including, but not limited to, V_{H} regions (V_{H}, V_{H}—V_{H}), anticalins, PepBodies, antibody-T-cell epitope fusions (Troybodies) or Peptibodies.

The term “monoclonal antibody” as used herein refers to an antibody from a population of substantially homogeneous antibodies, i.e., the individual antibodies comprising the population are substantially similar and bind the same epitope(s), except for possible variants that may arise during production of the monoclonal antibody, such variants generally being present in minor amounts. Such monoclonal antibody typically includes an antibody comprising a variable region that binds a target, wherein the antibody was obtained by a process that includes the selection of the antibody from a plurality of antibodies. For example, the selection process can be the selection of a unique clone from a plurality of clones, such as a pool of hybridoma clones, phage clones or recombinant DNA clones. It should be understood that the selected antibody can be further altered, for example, to improve affinity for the target, to humanize the antibody, to improve its production in cell culture, to reduce its immunogenicity in vivo, to create a multispecific antibody, etc., and that an antibody comprising the altered variable region sequence is also a monoclonal antibody of this invention. In addition to their specificity, the monoclonal antibody preparations are advantageous in that they are typically uncontaminated by other immunoglobulins. The modifier “monoclonal” indicates the character of the antibody as being obtained from a substantially homogeneous population of antibodies, and is not to be construed as requiring production of the antibody by any particular method. For example, the monoclonal antibodies to be used in accordance with the present disclosure may be made by a variety of techniques, including the hybridoma method (e.g., Kohler et al., Nature, 256:495 (1975); Harlow et al., Antibodies: A Laboratory Manual, (Cold Spring Harbor Laboratory Press, 2nd ed. 1988); Hammerling et al., in: Monoclonal Antibodies and T-Cell Hybridomas 563-681, (Elsevier, N. Y., 1981), recombinant DNA methods (see, e.g., U.S. Pat. No. 4,816,567), phage display technologies (see, e.g., Clackson et al., Nature, 352:624-628 (1991); Marks et al., J. Mol. Biol., 222:581-597 (1991); Sidhu et al., J. Mol. Biol. 338(2):299-310 (2004); Lee et al., J. Mol. Biol. 340(5): 1073-1093 (2004); Fellouse, Proc. Nat. Acad. Sci. USA 101(34): 12467-12472 (2004); and Lee et al. J. Immunol. Methods 284(1-2):119-132 (2004) and technologies for producing human or human-like antibodies from animals that have parts or all of the human immunoglobulin loci or genes encoding human immunoglobulin sequences (see, e.g., WO98/24893, WO96/34096, WO96/33735, and WO91/10741, Jakobovits et al., Proc. Natl. Acad. Sci. USA, 90:2551 (1993); Jakobovits et al., Nature, 362:255-258 (1993); Bruggemann et al., Year in Immune, 7:33 (1993); U.S. Pat. Nos. 5,545,806, 5,569,825, 5,591,669 (all of GenPharm); 5,545,807; WO 97/17852, U.S. Pat. Nos. 5,545,807; 5,545,806; 5,569,825; 5,625,126; 5,633,425; and 5,661,016, and Marks et al., Bio/Technology, 10: 779-783 (1992); Lonberg et al., Nature, 368: 856-859 (1994); Morrison, Nature, 368: 812-813 (1994); Fishwild et al., Nature Biotechnology, 14: 845-851 (1996); Neuberger, Nature Biotechnology, 14: 826 (1996); and Lonberg and Huszar, Intern. Rev. Immunol., 13: 65-93 (1995). Antibodies capable of specifically binding to an antigen of 5 to 12 amino acids comprising a sequence that (i) overlaps the junction (J) region and the constant (C) region of an immunoglobulin; or (ii) is within the first 15 residues from the C region of an immunoglobulin as defined herein can also be produced using phage display technology. Antibody fragments that selectively bind to the antigen defined herein can then be isolated. Exemplary methods for producing such antibodies via phage display are disclosed, for example, in U.S. Pat. No. 6,225,447.

The anti-CDRH3 peptide ligand, such as an anti-CDRH3 peptide antibody or antigen-binding fragment thereof, used for enrichment in the method described herein specifically binds to an epitope located in the junction (J) region and/or in the first 5 to 25, 20 or 15 residues from the constant (C) region of the immunoglobulin. The skilled person would understand that the antigen used to raise/select the anti-CDRH3 peptide ligand (e.g., antibody or antigen-binding fragment thereof) is selected based on the sequences of the immunoglobulin(s) of interest, which may be retrieved from suitable databases such as the international ImMunoGeneTics information system® (IMGT®. Sequences of the IGHJ regions and of the first residues from the IGHC region (ending at the conserved cysteine residue) from representative species (human, mouse and rhesus monkey) are depicted in Table 2 below (from IMGT®, Lefranc M-P, et al. Nucleic Acids Res. 2015 January; 43 (Database issue): D413-D422. Epub 2014 Nov. 5).

TABLE 2
Sequences of the IGHJ regions and of the first residues from the IGHC
region (ending at the conserved cysteine residue) from human, mouse
and rhesus monkey.

| Species | IGHJ sequences | IGHC sequences (first residues) |
|---|---|---|
| Human | J1: | IGHA1*01 (CH1): (SEQ ID NO:32) |
| | AEYFQHWGQGTLVTVSS | ASPTSPKVFPLSLCSTQPDGNVVIAC |
| | (SEQ ID NO: 26) | IGHA2*01 (CH1): (SEQ ID NO: 33) |
| | J2: | ASPTSPKVFPLSLDSTPQDGNVVVAC |
| | YWYFDLWGRGTLVTVSS | IGHD*01 (CH1): (SEQ ID NO: 34) |
| | (SEQ ID NO: 27) | APTKAPDVFPIISGCRHPKDNSPVVLAC |
| | J3: | IGHE*01 (CH1): (SEQ ID NO: 35) |
| | DAFDVWGQGTMVTVSS | ASTQSPSVFPLTRCCKNIPSNATSVTLGC |
| | (SEQ ID NO: 28) | IGHGP*01 (CH1): (SEQ ID NO: 36) |
| | J4: | ASTKGPSVFPLVPSSRSVSEGTAALGC |
| | YFDYWGQGTLVTVSS | IGHG1*01 (CH1): (SEQ ID NO: 37) |
| | (SEQ ID NO: 29) | ASTKGPSVFPLAPSSKSTSGGTAALGC |
| | J5: | IGHG2*01 (CH1): (SEQ ID NO: 38) |
| | NWFDSWGQGTLVTVSS | ASTKGPSVFPLAPCSRSTSESTAALGC |
| | (SEQ ID NO: 30) | IGHG3*01 (CH1): (SEQ ID NO: 39) |
| | J6: | ASTKGPSVFPLAPCSRSTSGGTAALGC |
| | YYYYYGMDVWGQGTTVTVSS | IGHG4*01 (CH1): (SEQ ID NO: 40) |
| | (SEQ ID NO: 31) | ASTKGPSVFPLAPCSRSTSESTAALGC |
| | | IGHM*01 (CH1): (SEQ ID NO: 41) |
| | | GSASAPTLFPLVSCENSPSDTSSVAVGC |
| | | IGHA1*01 (CH2): (SEQ ID NO: 42) |
| | | CCHPRLSLHRPALEDLLLGSEANLTC |
| | | IGHA2*01 (CH2): (SEQ ID NO: 43) |
| | | CCHPRLSLHRPALEDLLLGSEANLTC |
| | | IGHD*01 (CH2): (SEQ ID NO: 44) |
| | | ECPSHTQPLGVYLLTPAVQDLWLRDKATFTC |
| | | IGHE*01 (CH2): (SEQ ID NO: 45) |
| | | VCSRDFTPPTVKILQSSCDGGGHFPPTIQLLC |
| | | IGHGP*01 (CH2): (SEQ ID NO: 46) |
| | | TTEPLGGPSVFLFPPKPKDTLMISRTPEVTC |
| | | IGHG1*01 (CH2): (SEQ ID NO: 47) |
| | | APELLGGPSVFLFPPKPKDTLMI.SRTPEVTC |
| | | IGHG2*01 (CH2): (SEQ ID NO: 48) |
| | | APPVAGPSVFLFPPKPKDTLMISRTPEVTC |
| | | IGHG3*01 (CH2): (SEQ ID NO: 49) |
| | | APELLGGPSVFLFPPKPKDTLMISRTPEVTC |
| | | IGHG4*01 (CH2): (SEQ ID NO: 50) |
| | | APEFLGGPSVFLFPPKPKDTLMISRTPEVTC |
| | | IGHM*01 (CH2): (SEQ ID NO: 51) |
| | | VIAELPPKVSVFVPPRDGFFGNPRKSKLIC |
| | | IGHA1*01 (CH3): (SEQ ID NO: 52) |
| | | GNTFRPEVHLLPPPSEELALNELVTLTC |
| | | IGHA2*01 (CH3): (SEQ ID NO: 53) |
| | | GNTFRPEVHLLPPPSEELALNELVTLTC |
| | | IGHD*01 (CH3): (SEQ ID NO: 54) |
| | | AAQAPVKLSLNLLASSDPPEAASWLLC |
| | | IGHE*01 (CH3): (SEQ ID NO: 55) |
| | | DSNPRGVSAYLSRPSPFDLFIRKSPTITC |
| | | IGHGP*01 (CH3): (SEQ ID NO: 56) |
| | | GQPREPQVYTLPPSQKMTKNQVTLTC |
| | | IGHG1*01 (CH3): (SEQ ID NO: 57) |
| | | GQPREPQVYTLPPSRDELTKNQVSLTC |
| | | IGHG2*01 (CH3): (SEQ ID NO: 58) |
| | | GQPREPQVYTLPPSREEMTKNQVSLTC |
| | | IGHG3*01 (CH3): (SEQ ID NO: 59) |
| | | GQPREPQVYTLPPSREEMTKNQVSLTC |
| | | IGHG4*01 (CH3): (SEQ ID NO: 60) |
| | | GQPREPQVYTLPPSQEEMTKNQVSLTC |
| | | IGHM*01 (CH3): (SEQ ID NO: 61) |
| | | DQDTAIRVFAIPPSFASIFLTKSTKLTC |
| | | IGHE*01 (CH4): (SEQ ID NO: 62) |
| | | GPRAAPEVYAFATPEWPGSRDKRTLAC |
| | | IGHM*01 (CH4): (SEQ ID NO: 63) |
| | | GVALHRPDVYLLPPAREQLNLRESATITC |
| Mouse | J1: YWYFDVWGAGTTVTVSS | IGHA*01 (CH1): (SEQ ID NO: 68) |
| | (SEQ ID NO: 64) | ESARNPTIYPLTLPPVLCSDPVIIGC |
| | J2: YFDYWGQGTTLTVSS | IGHD*01 (CH1): (SEQ ID NO: 69) |
| | (SEQ ID NO: 65) | GDKKEPDMFLLSECKAPEENEKINLGC |
| | J3: WFAYWGQGTLVTVSA | IGHE*01 (CH1): (SEQ ID NO: 70) |
| | (SEQ ID NO: 66) | ASIRNPQLYPLKPCKGTASMTLGC |
| | J4: YYAMDYWGQGTSVTVSS | IGHG1*01 (CH1): (SEQ ID NO: 71) |
| | (SEQ ID NO: 67) | AKTTPPSVYPLAPGSAAQTNSMVTLGC |
| | | IGHG2A*01 (CH1): (SEQ ID NO: 72) |
| | | AKTTAPSVYPLAPVCGDTTGSSVTLGC |
| | | IGHG2B*01 (CH1): (SEQ ID NO: 73) |
| | | AKTTPPSVYPLAPGCGDTTGSSVTSGC |
| | | IGHG2C*01 (CH1): (SEQ ID NO: 74) |
| | | AKTTAPSVYPLAPVCGGTTGSSVTLGC |
| | | IGHG3*01 (CH1): (SEQ ID NO: 75) |
| | | ATTTAPSVYPLVPGCSDTSGSSVTLGC |
| | | IGHM*01 (CH1): (SEQ ID NO: 76) |
| | | ESQSFPNVFPLVSCESPLSDKNLVAMGC |
| | | IGHA*01 (CH2): (SEQ ID NO: 77) |
| | | SCQPSLSLQRPALEDLLLGSDASITC |
| | | IGHE*01 (CH2): (SEQ ID NO: 78) |
| | | VRPVNITEPTLELLHSSCDPNAFHSTIQLYC |
| | | IGHG1*01 (CH2): (SEQ ID NO: 79) |
| | | VPEVSSVFIFPPKPKDVLTITLTPKVTC |
| | | IGHG2A*01 (CH2): (SEQ ID NO: 80) |
| | | APNLLGGPSVFIFPPKIKDVLMISLSPIVTC |
| | | IGHG2B*01 (CH2): (SEQ ID NO: 81) |
| | | APNLEGGPSVFIFPPNIKDVLMISLTPKVTC |
| | | IGHG2C*01 (CH2): (SEQ ID NO: 82) |
| | | APDLLGGPSVFIFPPKIKDVLMISLSPMVTC |
| | | IGHG3*01 (CH2): (SEQ ID NO: 83) |
| | | PGNILGGPSVFIFPPKPKDALMISLTPKVTC |
| | | IGHM*01 (CH2): (SEQ ID NO: 84) |
| | | AVAEMNPNVNVFVPPRDGFSGPAPRKSKLIC |
| | | IGHA*01 (CH3): (SEQ ID NO: 85) |
| | | VNTFPPQVHLLPPPSEELALNELLSLTC |
| | | IGHD*01 (CH3): (SEQ ID NO: 86) |
| | | GAMAPSNLTVNILTTSTHPEMSSWLLC |
| | | IGHE*01 (CH3): (SEQ ID NO: 87) |
| | | DHEPRGVITYLIPPSPLDLYQNGAPKLTC |
| | | IGHG1*01 (CH3): (SEQ ID NO: 88) |
| | | GRPKAPQVYTIPPPKEQMAKDKVSLTC |
| | | IGHG2A*01 (CH3): (SEQ ID NO: 89) |
| | | GSVRAPQVYVLPPPEEEMTKKQVTLTC |
| | | IGHG2B*01 (CH3): (SEQ ID NO: 90) |
| | | GLVRAPQVYTLPPPAEQLSRKDVSLTC |
| | | IGHG2C*01 (CH3): (SEQ ID NO: 91) |
| | | GPVRAPQVYVLPPPAEEMTKKEFSLTC |
| | | IGHG3*01 (CH3): (SEQ ID NO: 92) |
| | | GRAQTPQVYTIPPPREQMSKKKVSLTC |
| | | IGHM*01 (CH3): (SEQ ID NO: 93) |
| | | SPSTDILTFTIPPSFADIFLSKSANLTC |
| | | IGHE*01 (CH4): (SEQ ID NO: 94) |
| | | GQRSAPEVYVFPPPEEESEDKRTLTC |
| | | IGHM*01 (CH4): (SEQ ID NO: 95) |
| | | EVHKHPPAVYLLPPAREQLNLRESATVTC |
| Rhesus | J1: AEYFEFWGQGALVTVSS | IGHA1*01 (CH1): (SEQ ID NO: 103) |
| monkey | (SEQ ID NO: 96) | PTKPKVFPLSLEGTQSDNVVVAC |
| (Macaca | J2: YWYFDLWGPGTPITISS | IGHD*01 (CH1): (SEQ ID NO: 104) |
| mulatta) | (SEQ ID NO: 97) | XDVFPIISACQLPKDNSPVVLAC |
| | J3: DAFDFWGQGLRVTVSS | IGHG1*01 (CH1): (SEQ ID NO: 105) |
| | (SEQ ID NO: 98) | ASTKGPSVFPLAPSSRSTSESTAALGC |
| | J4: YFDYWGQGVLVTVSS | IGHG2*01 (CH1): (SEQ ID NO: 106) |
| | (SEQ ID NO: 99) | SVFPLASCSRSTSQSTAALGC |
| | J5-1: NRFDVWGPGVLVTVSS | IGHG3*01 (CH1): (SEQ ID NO: 107) |
| | (SEQ ID NO: 100) | SVFPLASCSRSTSQSTAALGC |
| | J5-2: NSLDVWGQGVLVTVSS | IGHG4*01 (CH1): (SEQ ID NO: 108) |
| | (SEQ ID NO: 101) | SVFPLASSSRSTSESTAALGC |
| | J6: YYGLDSWGQGVVVTVSS | IGHM*01 (CH1): (SEQ ID NO: 109) |
| | (SEQ ID NO: 102) | GSASAPTLFPLVSCENAPLDTNEVAVGC |
| | | IGHA1*01 (CH2): (SEQ ID NQ: 110) |
| | | CDKPRLSLRRPALEDLLLGSEANLTC |
| | | IGHD*01 (CH2): (SEQ ID NO: 111) |
| | | ECPSHTQPLGVYLLPPALQDLWFQDKVTFTC |
| | | IGHG1*01 (CH2): (SEQ ID NO: 112) |
| | | APELLGGPSVFLFPPKPKDTLMISRTPEVTC |
| | | IGHG2*01 (CH2): (SEQ ID NO: 113) |
| | | AELLGGPSVFLFPPKPKDTLMI.SRTPEVTC |
| | | IGHG3*01 (CH2): (SEQ ID NO: 114) |
| | | APELLGGPSVFLFPPKPKDTLMISRTPEVTC |
| | | IGHG4*01 (CH2): (SEQ ID NO: 115) |
| | | APELLGGPSVFLFPPKPKDTLMISRTPEVTC |
| | | IGHM*01 (CH2): (SEQ ID NO: 116) |
| | | VLAERPPNVSVFVPPRDGFVGN.PRESKLIC |
| | | IGHA1*01 (CH3): (SEQ ID NO: 117) |
| | | GNTFRPEVHLLPPPSEELALNELVTLTC |
| | | IGHD*01 (CH3): (SEQ ID NO: 118) |
| | | AAQAPVRLSLNLLASSDPPEAASWLLC |
| | | IGHG1*01 (CH3): (SEQ ID NO: 119) |
| | | GQPREPQVYTLPPSREELTKNQVSLTC |
| | | IGHG2*01 (CH3): (SEQ ID NO: 120) |
| | | GQPREPQVYTLPPPREELTKNQVSLTC |
| | | IGHG3*01 (CH3): (SEQ ID NO: 121) |
| | | GQPREPQVYILPPPQEELTKNQVSLTC |
| | | IGHG4*01 (CH3): (SEQ ID NO: 122) |
| | | GQPREPQVYILPPPQEELTKNQVSLTC |
| | | IGHM*01 (CH3): (SEQ ID NO: 123) |
| | | NPDTAIRVFAIPPSFASIFLTKSTKLTC |
| | | IGHM*01 (CH4): (SEQ ID NO: 124) |
| | | GVAMHRPDVYLLPPAREQLNLRESATITC |

A combination of anti-CDRH3 peptide ligands, such as a combination of antibodies or antigen-binding fragments thereof, may also be used, for example to enrich for different subgroups of CDRH3 peptides (i.e. having different conserved epitopes in their sequences).

Thus, in another aspect, the present disclosure provides an anti-CDRH3 peptide ligand, such as an anti-CDRH3 peptide antibody or an antigen-binding fragment thereof, that specifically binds to an antigen of 5 to 15 amino acids comprising a sequence that (i) overlaps the J region and the C region of an immunoglobulin; or (ii) is within the first 25, 20, 15, 10 or 5 residues from the C region of an immunoglobulin.

In an embodiment, the immunoglobulin is a human or non-human primate immunoglobulin, preferably a human immunoglobulin. In an embodiment, the immunoglobulin is of the IgG class. In an embodiment, the immunoglobulin is a human IgG and the antigen comprises the sequence VTVSSASTK (SEQ ID NO:1, which corresponds to the last 5 residues of the human IgG J region and the first 4 residues of the human IgG constant region (CH1)). In an embodiment, the immunoglobulin is a human IgG and the antigen comprises the sequence GPSVFPLAP (SEQ ID NO:4, which corresponds to residues 5 to 13 of the human IgG constant region (CH1)) or ASTK(Me₂)GPSVFP (which corresponds to the first 10 residues of the human IgG constant region (CH1), with a dimethylated lysine).

In an embodiment, the anti-CDRH3 peptide antibody or antigen-binding fragment thereof is a polyclonal antibody. In another embodiment, the anti-CDRH3 peptide antibody or antigen-binding fragment thereof is a monoclonal antibody or antigen-binding fragment thereof. In a further embodiment, the anti-CDRH3 peptide antibody or antigen-binding fragment thereof comprises one of the following combinations of complementarity-determining regions (CDRs):

Combination 1

V_{H} CDR1: GFSLSSY (SEQ ID NO:5) or a variant thereof having one mutation;

V_{H} CDR2: DANDY (SEQ ID NO:6) or a variant thereof having one mutation;

V_{H} CDR3: YSRDGAIDPYFKI (SEQ ID NO:7) or a variant thereof having one mutation;

V_{L} CDR1: QSSQSVAGNRWAA (SEQ ID NO:8) or a variant thereof having one mutation;

V_{L} CDR2: QASKVTS (SEQ ID NO:9) or a variant thereof having one mutation; and

V_{L} CDR3: AGGYSGEFWA (SEQ ID NO:10) or a variant thereof having one mutation; or

Combination 2

V_{H} CDR1: GFSFSSGY (SEQ ID NO:11) ora variant thereof having one mutation;

V_{H} CDR2: DISGPY (SEQ ID NO:12) or a variant thereof having one mutation;

V_{H} CDR3: TDPTISSSYFNL (SEQ ID NO:13) or a variant thereof having one mutation;

V_{L} CDR1: QSSQSVYKNNRLA (SEQ ID NO:14) or a variant thereof having one mutation;

V_{L} CDR2: LASTLAS (SEQ ID NO:15) or a variant thereof having one mutation; and

V_{L} CDR3: QAYYDGYIWA (SEQ ID NO:16) or a variant thereof having one mutation.

In a further embodiment, the anti-CDRH3 peptide antibody or antigen-binding fragment thereof comprises one of the following combinations of CDRs:

Combination 1

V_{H} CDR1:
(SEQ ID NO: 5)
GFSLSSY;
V_{H} CDR2:
(SEQ ID NO: 6)
DANDY;
V_{H} CDR3:
(SEQ ID NO: 7)
YSRDGAIDPYFKI;
V_{L} CDR1:
(SEQ ID NO: 8)
QSSQSVAGNRWAA;
V_{L} CDR2:
(SEQ ID NO: 9)
QASKVTS;
and
V_{L} CDR3:
(SEQ ID NO: 10)
AGGYSGEFWA;

or

Combination 2

V_{H} CDR1:
(SEQ ID NO: 11)
GFSFSSGY;
V_{H} CDR2:
(SEQ ID NO: 12)
DISGPY;
V_{H} CDR3:
(SEQ ID NO: 13)
TDPTISSSYFNL;
V_{L} CDR1:
(SEQ ID NO: 14)
QSSQSVYKNNRLA;
V_{L} CDR2:
(SEQ ID NO: 15)
LASTLAS;
and
V_{L} CDR3:
(SEQ ID NO: 16)
QAYYDGYIWA.

In an embodiment, the anti-CDRH3 peptide antibody or antigen-binding fragment thereof comprises one of the following variable heavy chain (V_{H}) regions:

### V_{H}1

QSVEESGGRLVTPGTPLTLTCTVSGFSLSSYAMGWVRQAPGKGLEYIGIIDANDYIFYASWAKG RFTISKTSTTVDLKMTSPTTEDTATYFCARYSRDGAIDPYFKIWGPGTLVTVSS//GQPKAPSVF (SEQ ID NO:125), or a variant thereof having at least 70%, 80%, 90%, 95%, 96%, 97%, 98%, or 99% sequence identity.

### V_{H}2

QSLEESGGDLVKPGASLTLTCKASGFSFSSGYDICWVRQTPGKGLELIACIDISGPYTYYASWA KGRFTISKTSSTTVTLQLTSLTAADTATYFCAKTDPTISSSYFNLWGPGTLVTVSS//GQPKAPSV F (SEQ ID NO:126), or a variant thereof having at least 70%, 80%, 90%, 95%, 96%, 97%, 98%, or 99% sequence identity.

In an embodiment, the anti-CDRH3 peptide antibody or antigen-binding fragment thereof comprises one of the following variable light chain (V_{L}) regions:

### V_{L}1

QVLTQTPSPVSAALGGTVTINCQSSQSVAGNRWAAWYQQKSGQPPKLLIYQASKVTSGVPSR FSGSGSGTQFTLTISDLECDDAAIYYCAGGYSGEFWAFGGGTEVVVK//GDPVAPTVLLFPP (SEQ ID NO:127), or a variant thereof having at least 70%, 80%, 90%, 95%, 96%, 97%, 98%, or 99% sequence identity.

### V_{L}2

IDMTQTPSPVSAAVGDTVTISCQSSQSVYKNNRLAWYQQKPGQPPKLLIYLASTLASGVPSRFK GSGSGTQFTLTISEVQCDDAATYYCQAYYDGYIWAFGGGTEVWK//GDPVAPTVLLFPP (SEQ ID NO:128), or a variant thereof having at least 70%, 80%, 90%, 95%, 96%, 97%, 98%, or 99% sequence identity.

In an embodiment, the anti-CDRH3 peptide ligand, such as anti-CDRH3 peptide antibody or antigen-binding fragment thereof, is labelled or conjugated with one or more moieties. The anti-CDRH3 peptide ligand may be labeled with one or more labels such as a biotin label, a fluorescent label, an enzyme label, a coenzyme label, a chemiluminescent label, or a radioactive isotope label. In an embodiment, the anti-CDRH3 peptide ligand, such as anti-CDRH3 peptide antibody or antigen-binding fragment thereof, is labelled with a detectable label, for example a fluorescent moiety (fluorophore). Useful detectable labels include fluorescent compounds (e.g., fluorescein isothiocyanate (FITC), Texas red, rhodamine, fluorescein, Alexa Fluor® dyes, and the like), radiolabels, enzymes (e.g., horseradish peroxidase, alkaline phosphatase and others commonly used in a protein detection assays), streptavidin/biotin, and colorimetric labels such as colloidal gold, colored glass or plastic beads (e.g., polystyrene, polypropylene, latex, etc.). The anti-CDRH3 peptide ligand, such as anti-CDRH3 peptide antibody or antigen-binding fragment thereof, can also be conjugated to detectable or affinity tags that facilitate detection and/or purification of the ligand (e.g., antibody or antigen-binding fragment thereof). Such tags are well known in the art. Examples of detectable or affinity tags include polyhistidine tags (His-tags), polyarginine tags, polyaspartate tags, polycysteine tags, polyphenylalanine tags, glutathione S-transferase (GST) tags, Maltose binding protein (MBP) tags, calmodulin binding peptide (CBP) tags, Streptavidin/Biotin-based tags, HaloTag®, Profinity eXact® tags, epitope tags (such as FLAG, hemagglutinin (HA), HSV, S/S1, c-myc, KT3, T7, V5, E2, and Glu-Glu epitope tags), reporter tags such as β-galactosidase (β-gal), alkaline phosphatase (AP), chloramphenicol acetyl transferase (CAT), and horseradish peroxidase (HRP) tags (see, e.g., Kimple et al., Curr Protoc Protein Sci. 2013; 73: Unit-9.9).

In an embodiment, the anti-CDRH3 peptide ligand, such as anti-CDRH3 peptide antibody or antigen-binding fragment thereof, is bound to a solid support, such as beads (e.g., gel beads, resin beads, magnetic beads) or a polymer (monolithic column). The solid support may be conjugated with agents capable of binding to the anti-CDRH3 peptide antibody or antigen-binding fragment thereof such as anti-IgG, anti-IgA or anti-IgM antibodies, or certain proteins such as protein A or protein G (affinity immobilization). The anti-CDRH3 peptide ligand, such as anti-CDRH3 peptide antibody or antigen-binding fragment thereof, may alternatively be chemically attached to the solid support through primary amine, cysteine, carboxylic group, or sugar moiety, for example using 1-ethyl-3-(3-dimethylaminopropyl) carbodiimide hydrochloride (EDC) and N-hydroxysuccinimide (NHS). The solid support conjugated with the anti-CDRH3 peptide ligand, such as anti-CDRH3 peptide antibody or antigen-binding fragment thereof, may be incorporated into a chromatography column or used in suspension with beads (e.g., magnetic beads), for example, to isolate the CDRH3 peptide by affinity chromatography.

The antigen used to produce the anti-CDRH3 peptide antibody or antigen-binding fragment thereof may further comprise one or more modifications that confer additional biological properties to the antigen such as protease resistance, plasma protein binding, increased plasma half-life, intracellular penetration, etc. Such modifications include, for example, covalent attachment of molecules/moiety to the antigen such as fatty acids (e.g., C₆-C₁₈), attachment of proteins such as albumin (see, e.g., U.S. Pat. No. 7,268,113); sugars/polysaccharides (glycosylation), biotinylation or PEGylation (see, e.g., U.S. Pat. Nos. 7,256,258 and 6,528,485). The antigen may also be conjugated to a molecule that increases its immunogenicity, including vaccine carrier proteins such as keyhole limpet hemocyanin (KLH), bovine serum albumin (BSA), human serum albumin (HSA) and ovalbumin (OVA), and/or polysaccharides. In an embodiment, the peptide is conjugated to a carrier protein. In an embodiment, the vaccine carrier protein is conjugated via a disulfide bond to the antigen, i.e. through the sulfur of a cysteine residue of the antigen, for example a cysteine residue added at the N or C-terminal end of the antigen.

In another aspect, the present disclosure provides a composition comprising the antigen defined herein. In an embodiment, the composition further comprises the above-mentioned antigen and a carrier or excipient. Such compositions may be prepared in a manner well known in the pharmaceutical art.

In an embodiment, the composition is an immunogenic composition or vaccine composition. Such composition may be administered by any conventional route known in the vaccine field, e.g., via a mucosal (e.g., ocular, intranasal, pulmonary, oral, gastric, intestinal, rectal, vaginal, or urinary tract) surface, via a parenteral (e.g., subcutaneous, intradermal, intramuscular, intravenous, or intraperitoneal) route, or topical administration (e.g., via a transdermal delivery system such as a patch).

In an embodiment, the composition comprising the antigen defined herein further comprises a vaccine adjuvant. The term “vaccine adjuvant” refers to a substance which, when added to an immunogenic agent such as an antigen, non-specifically enhances or potentiates an immune response to the agent in the host upon exposure to the mixture. Suitable vaccine adjuvants are well known in the art and include, for example: (1) mineral salts (aluminum salts such as aluminum phosphate and aluminum hydroxide, calcium phosphate gels), squalene, (2) oil-based adjuvants such as oil emulsions and surfactant based formulations, e.g., incomplete or complete Freud's adjuvant, MF59 (microfluidised detergent stabilised oil-in-water emulsion), QS21 (purified saponin), AS02 [SBAS2] (oil-in-water emulsion+MPL+QS-21), (3) particulate adjuvants, e.g., virosomes (unilamellar liposomal vehicles incorporating influenza haemagglutinin), AS04 ([SBAS4] aluminum salt with MPL), ISCOMS (structured complex of saponins and lipids), polylactide co-glycolide (PLG), (4) microbial derivatives (natural and synthetic), e.g., monophosphoryl lipid A (MPL), Detox (MPL+M. Phlei cell wall skeleton), AGP [RC-529] (synthetic acylated monosaccharide), DC_Chol (lipoidal immunostimulators able to self-organize into liposomes), OM-174 (lipid A derivative), CpG motifs (synthetic oligonucleotides containing immunostimulatory CpG motifs), modified LT and CT (genetically modified bacterial toxins to provide non-toxic adjuvant effects), complete Freud's adjuvant (comprising inactivated and dried mycobacteria) (5) endogenous human immunomodulators, e.g., hGM-CSF or hIL-12 (cytokines that can be administered either as protein or plasmid encoded), Immudaptin (C3d tandem array) and/or (6) inert vehicles, such as gold particles.

In another aspect, the present disclosure provides a method for inducing the production of an antibody that specifically binds to the antigen described herein, in an animal, the method comprising administering to said animal an effective amount of the antigen described herein or composition comprising same defined herein to said animal. In another aspect, the present disclosure also provides the use of the antigen or composition comprising same defined herein for inducing the production of an antibody that specifically binds to the antigen in an animal.

In an embodiment, the above-mentioned method or use further comprises collecting the antibody produced in the animal. In a further embodiment, the above-mentioned method or use further comprises purifying the antibody collected using the epitope peptide used for immunization (e.g., CVTVSSASTK, SEQ ID NO:20) as a bait.

The animal to which the antigen or composition comprising same is administered may be any animal conventionally used for the production of antibodies, such as a rabbit, a guinea pig, a rat, a mouse, a goat, sheep or a chicken.

The complexes comprising the CDRH3 peptides and the anti-CDRH3 peptide antibody or antigen-binding fragment thereof is then dissociated to collect the CDRH3 peptides. Such dissociation may be achieved by any method known in the art, e.g. using appropriate reagents to disrupt the affinity interaction between the CDRH3 peptides and the anti-CDRH3 peptide antibody or antigen-binding fragment thereof. This may be achieved for example by lowering or raising the pH (acid or basic elution), by altering the ionic state of the solution (e.g., using a high salt solution) or by using chaotropic or denaturing agents (e.g., guanidine-HCl, ammonium thiocyanate, urea, SDS, etc.), or the use of organic solvents (methanol, acetonitrile) or any combinations of these approaches.

The eluted sample enriched in CDRH3 peptides may be subjected to any suitable treatment prior to LC-MS or any sequence analysis such as buffer exchange, concentration, dilution, etc. The CDRH3 peptides may also be modified, for example, using C-terminal and Asp/Glu modification with a group having a primary amine and a positive charge (e.g., methyl ester of arginine), which may increase charge state and thus reduce m/z and allow a longer stretch of amino acids to be sequenced), as described in PCT publication No. WO2020/124252. The CDRH3 peptides may also be digested with one or more endoproteases, e.g., using less specific enzymes such as pepsin or chymotrypsin or more specific enzymes such as Asp-N, Glu-C or Arg-C to obtain either shorter or longer peptides respectively.

In an embodiment, the method further comprises analyzing or characterizing the CDRH3 peptides. For example, the CDRH3 peptides could be resolved by reverse phase chromatography and in-line nanoelectrospray ionization/high-resolution tandem mass spectrometry, using well-established protocols to collect f tandem mass spectra from CDRH3 peptides. In an embodiment, the analyzing or characterizing is performed by mass spectrometry, preferably liquid chromatography-mass spectrometry (LC-MS).

In the methods described below, extraction of the MS/MS spectra associated to CDRH3 peptide was performed with a script to allow for the identification of spectra having specific ion signatures exclusively from the C-terminal, and more particularly the presence of 3 ions from the C-terminal end associated to the y6, y7, and y8 ions (ion signature triplet). However, the skilled person would understand that MS/MS spectra analysis may be performed using other methods and/or parameters including de novo peptide sequencing, or peptide sequencing using database search. In an embodiment, the analyzing or characterizing comprises determining the complete or partial amino acid sequence of the CDRH3 region of the CDRH3 peptide.

### EXAMPLES

The present technology is illustrated in further details by the following non-limiting examples.

### Example 1: Production of an Antibody Specific for the VTVSSASTK Antigen

Anti-CDRH3 peptide antibody (α-hCDRH3) generation. A rabbit polyclonal antibody was generated by New England Peptide (NEP), against the peptide “CVTVSSASTK” (SEQ ID NO:20), the N-terminal contains a cysteine residue in order to attach this peptide to a carrier protein to increase antigenicity and also to attach this peptide to a resin for the polyclonal antibody purification. The generated rabbit polyclonal antibody was purified against the antigen by New England Peptide and used as is (the antibody is hereinbelow referred to as “α-hCDRH3”).

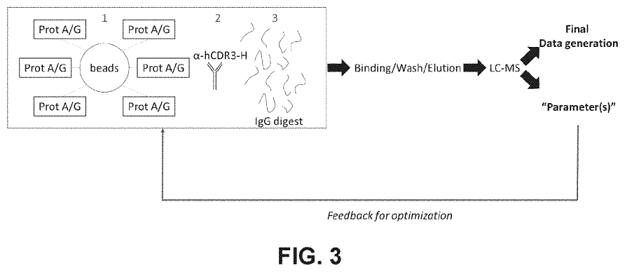

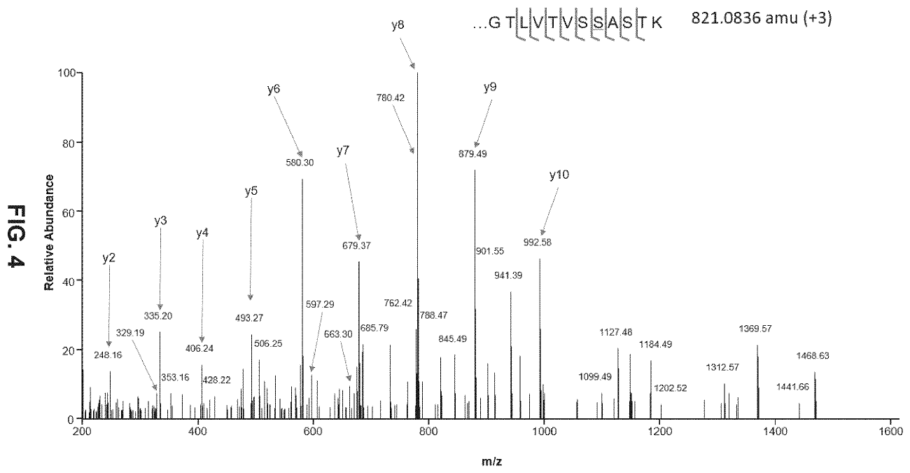

Generation of peptides from IgG—general procedure. Protein A or Protein G magnetic beads are incubated with the antibody α-hCDRH3 and washed and store at 4° C. A human plasma or an IgG human fraction is then reduced, alkylated and digested using a suitable lysine endoprotease (trypsin, Lys-C, or any other protease that generate meaningful peptide fragments that will include the CDRH3 and the targeted epitope from the J/C or C region). The protease is then inhibited using a protease inhibitor. A digest of few μg to several hundreds of μg and up to the low mg range has been used. The antibody α-hCDRH3 is then captured by affinity using protein A/G beads and incubated with the IgG peptide digest (see FIG. 3, items 1+2+3). The immune complexes are then washed with PBS with 0.03% CHAPS to remove any non-specific interactors. The hCDRH3 peptides are then eluted from the antibody with 0.1% formic acid in water followed by 0.1% TFA in 70% acetonitrile. The peptide mixture is then dried under low pressure (Speedvac®) and analyzed by LC-MS.

### Example 2: Enrichment of emCDRH3 Peptides from a First Plasma Sample (PD023I)

Plasma sample digestion. Plasma digestion was performed using a method similar to the one disclosed in Razavi et al., 2016. Small dry aliquot of denaturing buffer was made based on the method of Razavi et al (2016). 17 μL of a solution of 0.2 M tris, 9 M urea and 0.05M TCEP was dried. 10 μL of plasma (87 μg/μL of protein) was directly added to a dry aliquot of denaturing buffer and sonicated for 30 min and then another 30 min at room temperature (RT) to achieve reduction. 10 μL of a 0.1 M acrylamide solution was added, and then 115 μL of 0.2 M tris buffer and 5 μL of a 10 μg/μL trypsin solution were added before protein digestion was performed overnight. 20 μL of 100 mM PMSF was added to the digest to inhibit trypsin activity, followed by an incubation for at least 30 min before proceeding to the enrichment step.

Preparation of the protein G magnetic beads coupled to antibody anti-hCDR3. 25 μL of protein G slurry from Promega (Cat. No. G7471) was washed twice with 50 μL of phosphate buffer saline, PBS, pH 7.4 (composition is 137 mM NaCl, 2.7 mM KCl, 10 mM Na₂HPO₄, 1.8 mM KH₂PO₄). In parallel, 0.71 mL of the 0.349 μg/μL α-hCDRH3 antibody was washed in an Amicon 30 kDa MWCO (preparation split across 2 filters); the antibody was then centrifuged for 10 min before an additional 350 μL of PBS buffer was added. The PBS re-suspended antibody was then centrifuged for 20 min at 14,000 g. An additional 200 μL of PBS was added to extract the antibody (final volume of the antibody in 350 μL at 0.7 μg/μL). At this point, the α-hCDRH3 antibody was added to the washed protein G beads. The antibody-protein G beads complex was incubated for at least 30 min.

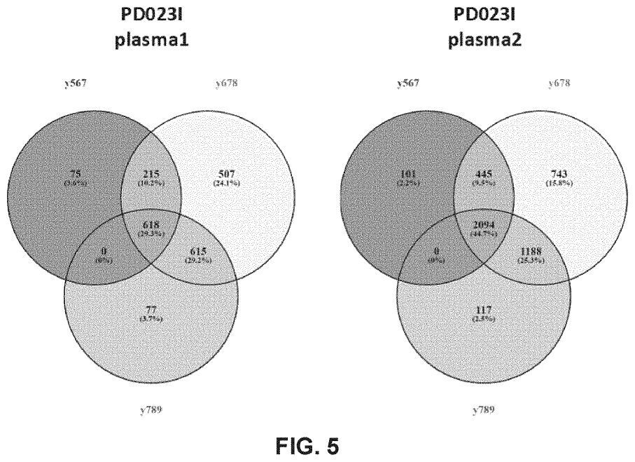

Immunoprecipitation (IP). 18 μL (refer to plasma 1 in FIG. 5) or 50 μL (refer to plasma 2 in FIG. 5) of the Protein G slurry coupled to α-hCDRH3 antibody was used on the 870 μg total protein digest and incubation overnight with constant mixing at room temperature. The IP complex was washed twice with 100 μL of PBS-0.03% CHAPS. The tube was then changed in order to reduce non-specific peptide interaction (i.e. presence of peptides bound to the tube). The solution-beads transfer was performed using a mix of PBS and CHAPS as the beads have a tendency to stick to tubes and pipette tips otherwise. The beads were washed twice with PBS-0.03% CHAPS (200 μl and 100 μl). For the first elution, 40 μL of 0.1% Formic Acid (FA)+10 μL acetonitrile were added, and after 5 min the supernatant was then transferred to a fresh collection tube. For the second elution, 100 μL of a 70% acetonitrile 0.1% trifluoroacetic acid (TFA) solution were added and incubated for 5 min, the supernatant was then transferred to the same collection tube as the first elution. The elution solution was dried under low vacuum (Speedvae). The dried sample was reconstituted in 0.1% FA in water and then 50% of the sample was loaded on an Evosep tip according to manufacturer's specifications.

LC-MS analysis. The LC-MS analysis was performed using an Evosep LC system connected to an Orbitrap fusion instrument (Thermo-Fisher). A 44 min LC-MS run was performed. Initial MS analysis was performed in HCD mode only with a mass range between 400 to 2000 amu. Charge states for the precursor were selected as at least 2+ and more, and all MS/MS spectra were acquired in centroid mode.

Data analysis was performed using a functionality from MS convert (MSConvertGUI), with one of the main parameters being the threshold defined as at least “150” of the most intense peaks within each MS/MS spectrum were kept for further analysis.

From all the spectra dataset, extraction of the spectra associated to emCDRH3 was performed with an in-house script to allow for the identification of spectra having specific ion signatures exclusively from the C-terminal (see FIG. 4 for typical MS/MS spectra having the conserved sequence of the targeted epitope).

The analysis was settled on the presence of 3 fragments for the automated detection of the emCDRH3 ions. It was found that using only 2 fragments generates too many false positives and that more than 3 fragments generates too many false negatives. The emCDRH3 peptides were enriched from 2 different plasma sample digests (plasma 1 and plasma 2) and the pool of MS/MS spectrum having a specific ion signature was compared. Different combinations of triplet ion signatures such as the different triplets y5,6,7 and y6,7,8 and y7,8,9 were compared (FIG. 5). A triplet is defined as the presence of those three specific fragments in a given MS/MS spectrum (with +/−50 ppm mass precision). A Venn diagram of the different spectra having a given triplet signature and their overlaps was generated. The combination y6,7,8 was the one generating the highest number of unique hits and the highest number of total hits (see FIG. 5) and was therefore used as the method to automatically count the number of hCDRH3 detected in an LC-MS run. The count of MS/MS spectra showing the ion signature y6 (580.2937 amu), y7 (679.3621 amu) and y8 (780.4098 amu) was used to roughly estimate the number of peptides having CDRH3 characteristics. Additional ions such as: y5: 493.2617 amu and y9: 879.4782 amu were also used (see FIG. 4).

The method efficacy to select hCDRH3 based on triplet of ion signature in a spectrum (i.e. the presence of 3 ions such as y6, y7 and y8) was manually inspected and supported by the presence of other typical emCDRH3 C-terminal ions not used in the selection criteria. The script allows to generate a list of the spectra containing the ion signature triplet including the parent ions mass, the scan number, the charge state and intensity.

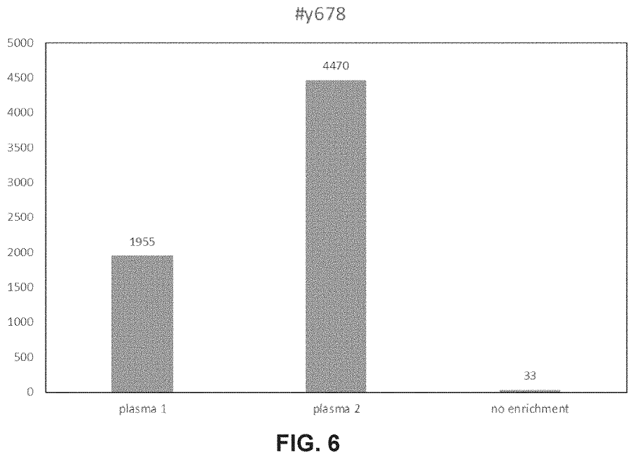

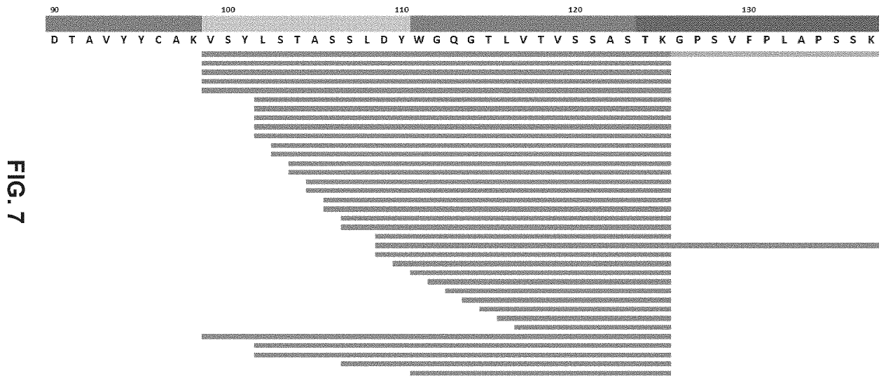

The data reported in FIG. 6 shows that the enrichment step using the antibody α-hCDRH3 permits to obtain an enrichment factor of 59 (when 18 μL of the protein G slurry beads was used, plasma 1) to 135 (when 50 μL of the protein G slurry beads was used, plasma 2) of the emCDRH3 sequences, providing compelling evidence that the use of the α-hCDRH3 antibody allows the successful enrichment of emCDRH3 peptides.

### Example 3: Enrichment of emCDRH3 Peptides Using Two Different Batches of α-hCDR3 Antibodies

Preparation of the Magnetic beads protein G coupled to antibody α-hCDRH3. A volume of 2×70 μL of protein G-beads slurry from Promega wash 2×200 μL with PBS-0.03% CHAPS. Two batches of antibodies α-hCDR3 obtained from the same rabbit were used (but from different bleeds):

Batch 1 α-hCDR3 abs 0.349 μg/μL 0.71 mL (250 μg)

Batch 2 α-hCDR3 abs 0.242 μg/μL 1 mL (250 μg)

250 μg of α-hCDR3 was added to the initial protein G volume of 70 μL. The α-hCDR3 antibody was used as is (i.e. without the wash step using the Amicon 30 kDa membrane cut-off). Overnight incubation was performed. The beads were washed three times with 250 μL PBS-0.03% CHAPS and reconstituted in 1 mL PBS-0.03% CHAPS before being used as is.

Plasma digestion: Human plasma with a total protein concentration of 87 μg/μL was used in these experiments. Similarly to example 1, 17 μL of a solution of 0.2 M tris, 9 M urea and 0.05 M tris(2-carboxyethyl)phosphine (TCEP) was dried. 10 μL of plasma (87 μg/μL of protein) was directly added to a dry aliquot of denaturing buffer and sonicated twice for 30 min at RT to achieve reduction. 15 μL of a 0.5 M iodoacetamide (IAA) solution, 115 μL of 0.2 M tris buffer, and 5 μL of a 10 μg/μL trypsin solution were added before digesting the protein overnight. 20 μL of a solution of 100 mM of phenylmethylsulfonyl fluoride (PMSF) was added to the digest to inhibit trypsin activity. The sample was incubated for at least 30 min prior to continuing to the enrichment step.

The IP was performed with both antibodies (batch 1 and batch 2) using the method described in Example 2. The enriched samples obtained were analyzed using an LC method on an Evosep LC pump as described in Example 2.

Data analysis generated the following number of counts of y6,7,8 ion signature:
Batch 1 α-hcdr3: 783 counts of y678
Batch 2 α-hcdr3-1204: 455 counts of y678.

These results show that both batches of antibodies led to an enrichment of the emCDRH3 peptides, with batch 1 leading to better enrichment.

### Example 4: Assessment of the Effect of Various Parameters on the Enrichment

In the next series of experiments, the effects of the following parameters on the levels of emCDRH3 peptide enrichment was assessed:
(1) Comparing protein A beads to protein G to immobilize antibody α-hCDRH3
(2) Comparing different digestion procedures, and
(3) Testing different Protein-G-α-hCDRH3 to plasma digest ratios.

(1) Protein A. Protein A coupled to magnetic beads was purchased from Promega. Protein A-α-hCDRH3 beads were prepared in a similar manner to Protein G-α-hCDRH3 (see Examples 2 and 3).

(2) Method 2 Digestion: 10 μL serum 87 μg/μL was mixed with 10 μL Guanidinium chloride (GuHCl) 6N and 10 μl 0.5 M TCEP and incubated at 95° C. for 15 min. 15 μL 0.5 M IAA was added at RT for 30 min. 55 μl of water was then added and 125 μL of 8 M urea was added before the pellet was sonicated. 25 μL 1M tetraethylammonium tetrahydroborate (TEAB) was added with 245 μL of water and 50 μg of trypsin. The digestion was performed overnight. 20 μL of a solution of 100 mM PMSF was added to the digest to inhibit trypsin activity. The sample was incubated for at least 30 min prior to continuing to the enrichment step.

(3) Different Protein G-α-hCDRH3 to plasma digest ratios. A control sample was used in similar conditions to Examples 2 and 3 with protein G-α-hCDRH3 (70 μL of the protein G-α-hCDRH3 slurry+870 μg plasma digest). A second sample which consists of more digest for the same amount of antibody (70 μL of protein G-α-hCDRH3+3×870 μg digest) was generated and a third sample which consists of more antibody for the same amount of digest (210 μl of protein G-α-hCDRH3+870 μg digest).

A significant enrichment of emHCDRH3 peptides was obtained under all conditions tested. A protocol using protein A instead of protein G to capture α-hCDRH3 and capture emHCDRH3 peptides by IP generates a smaller number (about 2-times less, 757 vs. 1526) of MS/MS spectra with the y6,7,8 ions of a emHCDRH3 peptide, which was unexpected since Protein A has been reported to bind preferably rabbit IgG compared to Protein G.

### Example 5: Assessment of the Effect of Plasma Digest Cleanup on the Enrichment

The main objective of this experiment was to determine if cleanup of the plasma digest on reverse phase in order to both reduce the volume and remove GuHCl and urea (which could interfere with the antigen sequence binding of the α-hCDRH3) would improve the enrichment of emHCDRH3 peptides. Human plasma (87 μg/μL) and digestion method 2 were used in this experiment.

After overnight digestion, the digestion mixture was cleaned up using Bond Elut LMS (Agilent) 25 mg beads volume. All operated with gravity flow, 1 mL Methanol followed by 2×1 mL water then the entire digest sample was loaded and washed with 1 mL water and then eluted with 1 mL acetonitrile. Samples were dried under low pressure. Four different sample experiments were performed using the antibody of batch 2.
Condition 1) Control (i.e. no SPE cleanup, using digest method 2 and similar IP procedure to example 1)
Condition 2) Use an equivalent of 1× 870 μg digest plasma with 50 μL protein G-Beads-batch 2 antibody
Condition 3) Use an equivalent of 3× 870 μg digest plasma with 50 μL G-Beads-batch 2 antibody
Condition 4) Use an equivalent of 3× 870 μg digest plasma with 2× 50 μL G-Beads-batch 2 antibody

The results obtained under these four conditions are as follows:
Condition 1) 7283 MS/MS spectra with y6,7,8
Condition 2) 9206 MS/MS spectra with y6,7,8
Condition 3) 4332 MS/MS spectra with y6,7,8
Condition 4) 5425 MS/MS spectra with y6,7,8

These results suggest that sample cleanup helps significantly (comparing conditions 1 and 2). Under these conditions, changing the beads/digest ratio did not increase the number of MS/MS having the y6,7,8 ion signature of a hCDRH3. Condition 1 was also tested with the batch 1 antibody and about 10,214 MS/MS with the specific y6,7,8 ions were identified.

### Example 6: Enrichment of emCDRH3 Peptides from an Antibody Standard

The Promega standard protein antibody (IgG1, Cat. No. CS302902) was reduced, alkylated and digested with 2 enzymes (trypsin and Asp-N). For the enrichment with the protein G-α-hCDRH3, 12.5 μg of each digest was used, with 20 μL of the protein G-α-hCDRH3 slurry (the slurry was washed twice with 100 μL PBS-0.03% CHAPS). Antibody from batch 1 was used.

The Promega antibody digest was added to the beads with 37.5 μL H₂O, and incubated overnight. The supernatant was removed and beads were washed twice with 100 μL PBS-0.03% CHAPS, before transferring to a new tube then washing with 200 μL PBS-0.03% CHAPS followed with 100 μL PBS-0.03% CHAPS. For the first elution, 40 μL of 0.1% Formic Acid (FA)+10 μL acetonitrile were added, and after 5 min the supernatant was transferred to a fresh collection tube. For the second elution, 100 μL of a 70% acetonitrile 0.1% trifluoroacetic acid (TFA) solution were added, and after 5 min the supernatant was transferred to the same collection tube as the first elution. The elution solution was dried under low vacuum (Speedvae). Samples of both digests as well as a sample without enrichment were run for comparison.

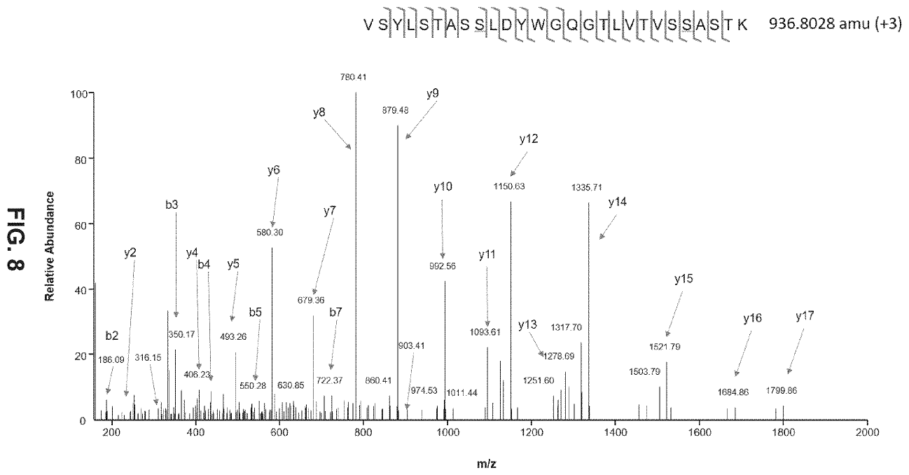

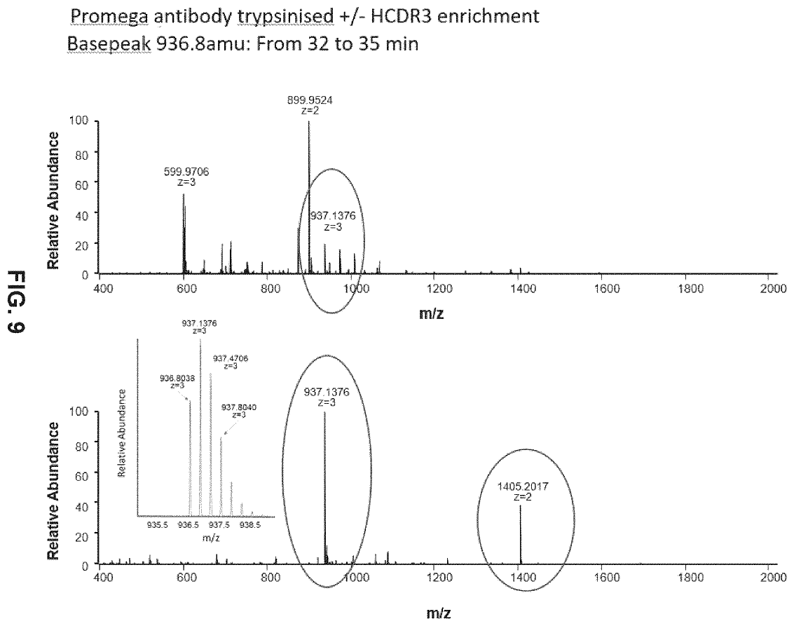

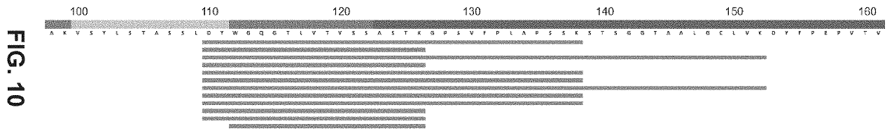

The elution solution was dried under low vacuum (Speedvac®) and reconstituted in 7 μL 0.1% FA in water and loaded on an Easy nLC 1000, 15 cm column RP (pepMAP RSLC C18, 3 μm 100A 75 μm×15 cm) the samples were run on an Orbitrap Fusion Lumos. Most data were acquired in HCD mode or EThcD mode when specified. Without enrichment, both trypsin and Asp-N generate a good coverage of the Promega antibody. Analysis of the enrichment strategy with trypsin is shown in FIG. 7, highlighting a significant enrichment of the tryptic peptides containing the VTVSSASTK sequence. Several peptides from that region were identified with very little coverage of other sequences of that antibody. In FIG. 8, an MS/MS spectrum of the emCDRH3 of the Promega antibody peptide is shown with a good coverage of both C and N terminal fragments. A good coverage of the peptide sequence is shown confirming this peak at 936.80278 amu as a 3+ is indeed associated to a peptide containing both the CDRH3 region and the targeted epitope VTVSSASTK (SEQ ID NO:1). The overall elution window of that peptide (between 32 min and 35 min) is shown in the case of an entire, non-enriched tryptic digest (FIG. 9, upper part) versus enriched (FIG. 9, bottom part). Following enrichment, the dominating peptides are the 936.80278 and 1404.701264 amu which are the 3+ and 2+ charge state forms of the sequence VSYLSTASSLDYWGQGTLVTVSSASTK (SEQ ID NO:17) respectively.

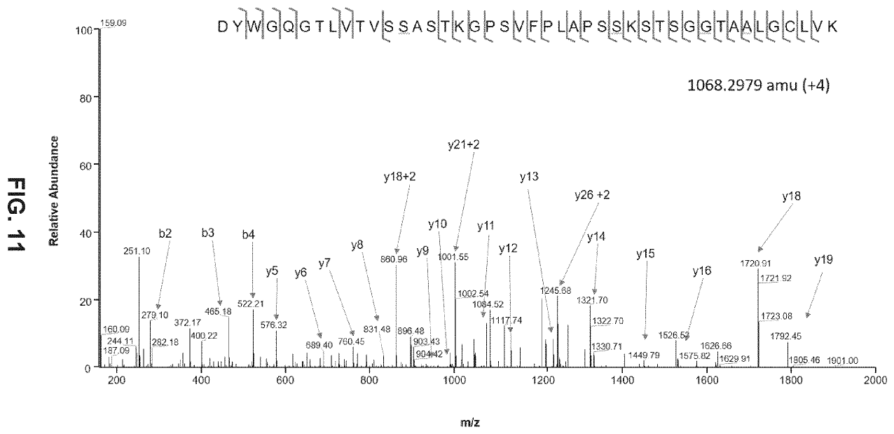

It was next tested if it was possible to enrich peptides resulting from digestions other than trypsin where the epitope sequence VTVSSASTK is in the middle of a sequence and not necessarily at the C-terminal end. The concerned case applies to an Asp-N digest, which does not cleave after a lysine residue like trypsin. Most of the enriched peptides shown in FIG. 10 contain the VTVSSASTK sequence. One of the targeted peptides, DYWGQGTLVTVSSASTKGPSVFPLAPSSKSTSGGTAALGCLVK (SEQ ID NO:129) is a 4+ peptide with an apparent mass of 1068.29796 amu (shown in FIG. 11 to confirm the sequence), thus confirming that the approach is suitable to enrich peptides in which the targeted epitope is not at the C-terminal end.

### Example 7: Enrichment of emCDRH3 Peptides from Other Species

Samples were prepared as described above in Example 5. Plasma used in these experiments was purchased from Creative Biolabs NHP Biologicals. Blood from Rhesus monkey (Macaca mulatta) and the crab-eating macaque (Macaca fascicularis) was used. 10 μL of plasma was used with digestion method no 2. Enrichment was performed as described above using protein G beads using batch 1 antibody. The immunoprecipitation procedure is the same as used in Example 2. The samples were analyzed in an Orbitrap Fusion Lumos instrument with a 15 cm RP column run on an easy nLC 1000 LC system. All samples were run in HCD mode.

Below are the numbers of spectra containing y6,7,8 ion signature of an emhCDRH3 peptides in each sample.

M. mulatta: 2613 MS/MS with y6,7,8

M. fascicularis: 3039 MS/MS with y6,7,8

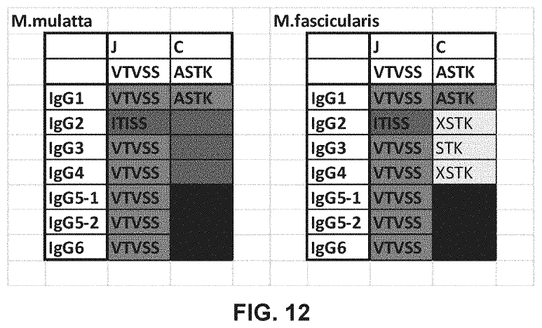

In the case of H. sapiens, the VTVSSASTK is conserved across all IgG while for M. mulata and M. fascicularis, this sequence is only conserved in IgG1 and partly in other isoforms (see FIG. 12, generated from information extracted from the international ImMunoGeneTics information system®, IMGT). For example, the emhCDRH3 peptide sequences APPGNVADSWGQGVLVTVSSASTK (SEQ ID NO:130) and FDVWGPGVLVTVSSASTK (SEQ ID NO:131) were identified in the M. mulata plasma, and the emhCDRH3 peptide sequence FWDVWGPGVLVTVSSASTK (SEQ ID NO:132) was identified in M. fascicularis plasma.

The detection of several MS/MS spectra indicating the presence of the VTVSSASTK epitope in the two species of monkeys shows that the enrichment strategy works on species other than H. sapiens.

### Example 8: IgG Enrichment from Plasma Sample Using Digestion from the Protein G-Beads Followed by an Enrichment of the emCDRH3 Peptides

The objective of these experiments is to enrich IgG first from dilute samples (in this case, plasma; however, any other fluid that contains IgG may also be used). Instead of eluting the IgG from the G-beads, which may result in a poor elution efficiency, a complete protease digestion of the mix protein-G-beads and antibody was done followed by an enrichment of the emCDRH3 peptides. The experiments were performed on a 10 μL and 50 μL plasma sample (plasma1 at 87 μg/μL). 20 μL and 70 μL of protein G magnetic beads slurry from Promega was used (G7471) and washed twice with 500 μL PBS. 90 μL PBS-0.03% CHAPS (PBS-CHAPS) was added to 10 μL plasma which was added to the 20 μL G-beads slurry. 50 μL PBS-CHAPS to 50 μL plasma was added to the 70 μL G-beads slurry, followed by incubation for 1 h with tumbling. The supernatant was removed and the beads were rinsed twice with 100 μL PBS-CHAPS followed by 100 μL PBS add 5 μL DTT (0.5M)+50 μL water 95° C. for 15 min; 15 μL of 0.5 M IAA was added and incubated at 37° C. for 1 h. 25 μL of 1M TEAB, 280 μL H₂O, 125 μL 8M urea and 5 μL of trypsin 10 μg/μL was added then digested overnight. 20 μL of 100 mM PMSF was added to stop protease activity (incubation 30 min). The digest was cleaned using 25 mg Bond Elut LMS SPE column (Agilent). 500 μL MeOH followed with 2×1 mL water conditioning, the samples were loaded on the SPE column, washed with 1 mL water then eluted with 200 μL MeOH followed by 1 mL ACN.

The samples were dried under low pressure and reconstituted into 100 μL PBS-CHAPS, added to 50 μL G-beads-α-hCDRH3 (batch 1 antibody). The rest of the procedure is as described in Example 6. Samples were analyzed on an Evosep-Fusion instrument in HCD mode. As shown below, the counts of y6,7,8 was good for both samples but higher in the 10 μL plasma relative to the 50 μL plasma, suggesting that the non-specific interactors were reduced for the 10 μL plasma.
10 μL plasma: total y6,7,8: 6840 MS/MS spectra
50 μL plasma: total y6,7,8: 5754 MS/MS spectra

### Example 9: IgG Enrichment from Saliva Using G-Beads Followed by CDRH3 Enrichment Using α-hCDRH3 Immunoprecipitation

Approximately 2×1 mL of saliva was collected from a single donor, a 2×20 μL of protein G magnetic beads slurry (Promega) was washed twice with 500 μL of PBS and 0.03% CHAPS and the 20 μL equivalent washed beads were added to the 1 mL saliva and mixed overnight (2 tubes of 1 mL saliva is then processed in parallel). Beads were washed twice with 500 μL PBS-CHAPS, the third wash, done as well with 500 μL PBS-CHAPS before transferring to a fresh tube followed by a 200 μL wash with only PBS and removed. To the beads, 50 μL water+5 μL 1M dithiothreitol (DTT) was added, incubated at 95° C. for 15 min, and 15 μL 0.5M IAA was added followed by incubation of 1 h at RT. 125 μL 8M urea, 25 μL 1M TEAB+280 μL water were added, followed by addition of 50 μg trypsin for a 37° C. overnight digest. 20 μL 100 Mm PMSF was added to stop protease activity. The peptide digest was cleaned on Bond Elut LMS (Agilent) as described in Example 7. After being dried down, the sample was reconstituted into 100 μL PBS-CHAPS and added to a slurry of 50 μL G-beads-α-hCDRH33 (batch 2 antibody). The mixture was incubated overnight, and the rest of the procedure was conducted similarly to Example 6.

A total of 4193 MS/MS spectra with the signature y6,7,8 were found, including the IgG h-CDR3 peptide sequence WFDPWGQGTLVTVSSASTK (SEQ ID NO:133).

### Example 10: Sequencing of the 2 Highly Represented Antibodies Present in the Rabbit Polyclonal α-hCDRH3

The main IgG components of the rabbit polyclonal α-hCDRH3 antibody used in the studies described herein to enrich CDRH3 peptides containing the sequence VTVSSASTK which is the C-terminal part of the J/C peptide following a trypsin or Lys-C digestion. The rabbit polyclonal antibody was raised and purified against the antigen by New England peptides, different batches of antibodies were collected, for the second batch, an aliquot of blood was collected, and B-cell sequencing was performed by Genewiz. One hundred ug of rabbit antibody α-hCDRH3 was reduced with dithiothreitol and alkylated with iodoacetamide, precipitated in acetone and reconstitute into a small amount of 4M urea. The samples were separated into 5 tubes and digested with 5 different proteases: trypsin, LysC, AspN, chymotrypsin and pepsin. In addition, the rabbit polyclonal anti-hCDRH3 was also separated on hydrophobic interaction chromatography and on native gel. Different protein fractions were then digested with trypsin and chymotrypsins, all of the different peptide extracts were analyzed by LC-MS, the MS/MS spectra were analyzed and antibody assembling was also performed using B-Cell repertoire. Several antibodies were paired and assembled. Four paired H/L IgG antibody sequences were identified and send to Sino Biological for synthesis. The 4 IgG were tested for affinity using an ELISA strategy against the VTVSSASTK peptide and 2 of the 4 IgG (named PD030_r1 and PD030_r3) were shown to have a higher affinity than that of the polyclonal antibody. The sequences and their associated CDR are the following:

PD030 r1
V_{H}
(SEQ ID NO: 125)
QSVEESGGRLVTPGTPLTLTCTVSGFSLSSYAMGWRQAPGKGLEYIGII
DANDYIFYASWAKGRFTISK
TSTTVDLKMTSPTTEDTATYFCARYSRDGAIDPYFKIWGPGTLVTVSS
//GQPKAPSVF...
V_{L}
(SEQ ID NO: 127)
QVLTQTPSPVSAALGGTVTINCQSSQSVAGNRWAAWYQQKSGQPPKLLI
YQASKVTSGVPSRFSGSGSGT
QFTLTISDLECDDAAIYYCAGGYSGEFWAFGGGTEWVK//
GDPVAPTVLLFPP...
V_{H} CDR1:
(SEQ ID NO: 5)
GFSLSSY
V_{H} CDR2:
(SEQ ID NO: 6)
DANDY
V_{H} CDR3:
(SEQ ID NO: 7)
YSRDGAIDPYFKI
V_{L} CDR1:
(SEQ ID NO: 8)
QSSQSVAGNRWAA
V_{L} CDR2:
(SEQ ID NO: 9)
QASKVTS
V_{L} CDR3:
(SEQ ID NO: 10)
AGGYSGEFWA
PD030 r3
V_{H}
(SEQ ID NO: 126)
QSLEESGGDLVKPGASLTLTCKASGFSFSSGYDICWVRQTPGKGLELIA
CIDISGPYTYYASWAKGRFTI
SKTSSTTVTLQLTSLTAADTATYFCAKTDPTISSSYFNLWGPGTLVTVS
S//GQPKAPSVF...
V_{L}
(SEQ ID NO: 128)
IDMTQTPSPVSAAVGDTVTISCQSSQSVYKNNRLAWYQQKPGQPPKLLI
YLASTLASGVPSRFKGSGSGT
QFTLTISEVQCDDAATYYCQAYYDGYIWAFGGGTEWVK//GDPVAPTVL
LFPP...
V_{H} CDR1:
(SEQ ID NO: 11)
GFSFSSGY
V_{H} CDR2:
(SEQ ID NO: 12)
DISGPY
V_{H} CDR3:
(SEQ ID NO: 13)
TDPTISSSYFNL
V_{L} CDR1:
(SEQ ID NO: 14)
QSSQSVYKNNRLA
V_{L} CDR2:
(SEQ ID NO: 15)
LASTLAS
V_{L} CDR3:
(SEQ ID NO: 16)
QAYYDGYIWA

### Example 11: Comparing the Performance of the Natural Rabbit Polyclonal Antibodies (pAbs) with the 2 Recombinant Forms Identified in the pAbs Mixture

Two hundred μg of the Promega standard protein antibody (IgG1, Cat. No. CS302902) was reduced, alkylated and digested with trypsin. 1 μL 100 mM PMSF inhibitor was added. For the enrichment with the protein G-α-hCDRH3, 12.5 μg of the digest was used per experiment, with 20 μL the protein G-α-hCDRH3 slurry preparation is described below. Twenty μg of antibody from batch 2 (PD030) was used and twenty μg of 4 recombinant antibodies PD030_r1, PD030_r2, PD030_r3 and PD030_r4 were coupled to 10 μL of protein G beads from Promega (Cat. No. G7471) by tumbling at room temperature for 1 hour. The supernatant was removed, then the beads were washed three times with 100 μl PBS 0.03% CHAPS. The protein G-α-hCDRH3 slurry was then diluted to 80 μL using PBS 0.03% CHAPS to create a stock solution. A negative control rabbit IgG was used (named “IgG”), two of the recombinant non-binders were also used (PD030_r2 and PD030_r4), and the two recombinant binder form were used as well (PD030_r1 and PD030_r3). The Promega standard antibody digest (12.5 μg) was added to the beads with 12.5 μL PBS 0.03% CHAPS, and incubated for 1 hour by tumbling at room temperature. The supernatant was removed, and beads were washed twice with 100 μL PBS-0.03% CHAPS, followed by 100 μL PBS. For the first elution, 40 μL of 0.1% Formic Acid (FA)+10 μL acetonitrile were added, and after 5 min the supernatant was transferred to a fresh collection tube. For the second elution, 100 μL of a 70% acetonitrile 0.1% trifluoroacetic acid (TFA) solution were added, and after 5 min the supernatant was transferred to the same collection tube as the first elution. The elution solution was dried under low vacuum (Speedvae). Samples of both digests as well as a sample without enrichment were run for comparison. The dried samples were reconstituted in 0.1% FA in water and then 100% of the sample was loaded on an Evosep tip according to manufacturer's specifications. The LC-MS analysis was performed using an Evosep LC system connected to an Orbitrap QExactive instrument (Thermo-Fisher). A 44 min LC-MS run was performed. Initial MS analysis was performed in HCD mode only with a mass range between 400 to 2000 amu. Charge states for the precursor were selected as at least 2+ and more, and all MS/MS spectra were acquired in centroid mode.

As in Example 6, a good coverage of the peptide sequence was found confirming this peak at 936.80278 amu as a 3+ is indeed associated to a peptide containing both the CDRH3 region and the targeted epitope VTVSSASTK (with the sequence VSYLSTASSLDYWGQGTLVTVSSASTK). Basepeak extraction at 936.80278 amu was performed for all immunoprecipitation, Area under the peak was extracted and normalized against the same peaks found in the sample “IgG” negative control. For the 2 recombinant forms having no detectable affinity for the VTVSSASTK sequence, (PD030_r2 and PD030_r4), the area under the curve normalized against the IgG negative control show value of 0.7 and 0.6 respectively. However, for the rabbit natural pAbs (PD030), this ratio was found at 11.7 while for the 2 monoclonal antibody showing an affinity against the VTVSSASTK epitope, the area under the curve for the 936.80278 amu as a 3+ is at 40 and 32 for PD030_r1 and PD030_r3, respectively, showing an higher efficiency to enrich for the targeted epitope relative to the polyclonal Abs.

### Example 12: Enrichment of CDRH3 Fragments from a Subject Previously Tested Positive for Coronavirus Disease 19 (COVID-19)

For this series of experiments, plasma from the Oklahoma blood bank, more specifically from a volunteer male (67 years old) who previously tested positive for COVID-19, was used.

The recombinant form of the PD030_r1 (R1) antibody described above, a rabbit IgG antibody that recognise the epitope “VTVSSASTK” that is often present in the J/C region of human heavy CDR3, was used. 250 μg of the recombinant antibody R1 was coupled with 70 μL magnetic protein G slurry (Magne® protein G beads Promega). Initially the 70 μL of protein G slurry was washed twice with 500 μL 0.01M PBS 0.03% CHAPS. The solution antibody R1 and protein G beads was completed up to 100 μL using 0.01M PBS 0.03% CHAPS then gently mixed at room temperature for 1 hour. The supernatant was removed, and the magnetic beads were washed three times with 500 μL 0.01M PBS 0.03% CHAPS to remove any unbound R1. A stock solution of antibody R1 coupled to protein G beads was then made in 1 mL 0.01M PBS 0.03% CHAPS.

The following four conditions were used:
1. 20 μL plasma with standard enrichment method with urea and Reverse phase solid phase extraction LMS cleanup (RP-SPE);
2. 20 μL plasma with a method using less urea and no RP-SPE.
3. 100 μg IgG enriched using protein G, standard enrichment methods with RP-SPE cleanup
4. 100 μg IgG enriched using protein G with less urea and no RP-SPE clean-up

Condition 1 and 2: Sample Preparation (IgG Digest on Protein G Magnetic Beads) 40 μL of magnetic protein G slurry was washed twice with 500 μL 0.01M PBS 0.03% CHAPS. 20 μL of plasma was added, and the mixture was diluted up to 100 μL using 80 μL 0.01M PBS 0.03% CHAPS and mixed at room temperature for 1 hour. The supernatant was removed from the protein G-IgG magnetic beads, which were washed twice with 100 μL 0.01M PBS 0.03% CHAPS and once with 100 μL 0.01M PBS and removed supernatant.

Condition 1: 50 μL of water was added followed by 5 μL of 1M DTT. The mixture was incubated at 95° C. for 15 minutes, and the tubes were allowed to cool to room temperature. 15 μL of 0.5M lodoacetamide (IAA) was then added followed by a 1-hour incubation in the dark. 25 μL of 1M triethylammonium bicarbonate (TEAB Sigma T7408), 125 μL of 8M urea, 280 μL of HPLC grade water and 5 μL of 50 μg/μl trypsin (Worthington cat LS3003703) were added, and the trypsin digestion was performed overnight at 37° C. The leftover magnetic beads were set apart from the solution using a magnetic rack and the supernatant was transferred to a new tube. The digest solution was cleaned on a Bond Elut LMS cartridge, 25 mg, 1 mL (Agilent) following manufacturer's suggested procedure and the eluate was dried under low pressure centrifuge.

Condition 2: 50 μL of water was added followed by 1.5 μL of 1M DTT. The mixture was incubated at 95° C. for 15 minutes, and the tubes were allowed to cool to room temperature. 5 μL of 0.5M lodoacetamide (IAA) was then added followed by a 30-minute incubation in the dark. 46 μL of 100 mM TEAB, 10 μL of 4M urea, and 5 μL of trypsin (sequencing grade modified Promega) were added, and the trypsin digestion was performed overnight at 37° C. The digest solution was dried under low pressure centrifuge.

Condition 3 and 4: sample preparation from IgG Enrichment from Plasma using Agarose Protein G

6 mL of agarose protein G slurry (3 mL settled Agarose beads, Genscript Protein G resin cat no L00209) was added to an empty 20 mL Blared® column (Econo-Pac® Chromatography Column, #7321010 polypropylene columns 20 mL bed volume). The Protein G column was conditioned by passing four times 15 mL 0.01M PBS. 4 mL of plasma was centrifuged at 23,000×g for 10 minutes at 4° C. To this 4 mL of plasma, 8 mL of 0.01M PBS was added then filtered using Millex® low protein bind filter (0.45 um PVDF HV cat no SLHVRO4NL Millipore®) using a syringe. The filtered plasma was loaded and passed over the protein G column three times. The protein G column was washed three times with 10 mL 0.01M PBS. IgG fraction was eluted by adding 12.5 mL 0.1M glycine buffer pH 2.7 to beads, the elution was collected in a 15 mL Falcon® tube containing 2.5 mL 1M tris pH 8 to neutralise the glycine elution. The IgG solution was concentrated using a Amicon® Ultra—4, 30 kDa molecular cut-off (cat UFC803024), the solution was centrifuged at 2400×g with 10-minute intervals allowing top up of the IgG solution to concentrate, then the buffer was exchanged using 0.01M PBS. The final concentration of IgG was 16.989 mg/mL with a final volume of 1.1 mL (for 4×1 ml of plasma).

100 μg of IgG enriched from plasma using agarose protein G was dried down (centrifuge under low pressure). Samples were reconstituted in 50 μL water.

Condition 3: 5 μL of 1M DTT was added. The mixture was incubated at 95° C. for 15 minutes, and the tubes were allowed to cool to room temperature. 15 μL of 0.5M IAA was then added followed by a 1-hour incubation in the dark. 25 μL of 1M triethylammonium bicarbonate (TEAB Sigma T7408), 125 μL of 8M urea, 280 μL of HPLC grade water and 5 μL of 50 μg/μL trypsin (Worthington cat LS3003703) were added, and the trypsin digestion was performed overnight at 37° C. The leftover magnetic beads were set apart from the solution using a magnetic rack and the supernatant was transferred to a new tube. The digest solution was cleaned on a Bond Elut LMS cartridge, 25 mg, 1 mL (Agilent) following manufacturer's suggested instructions and the eluate was dried under low pressure centrifuge (Speedvae).

Condition 4: 1.5 μL of 1M DTT was added. The mixture was incubated at 95° C. for 15 minutes, and the tubes were allowed tubes to cool to room temperature. 5 μL of 0.5M IAA was then added followed by a 30-minute incubation in the dark. 46 μL of 100 mM TEAB, 10 μL of 4M urea, and 5 μL of trypsin (sequencing grade modified Promega) were added, and the trypsin digestion was performed overnight at 37° C. The digest solution was dried under low pressure centrifuge (Speedvae).

50 μl of the stock solution magnetic beads protein G-R1 antibody was used per immunoprecipitation. The supernatant was removed. Each sample from condition 1 to 4 were reconstituted into 100 μL 0.01M PBS 0.03% CHAPS plus 1 μL of 100 mM of the trypsin inhibitor phenylmethanesulfonyl fluoride (PMSF) (final inhibitor concentration of 1 mM) and was added to the magnetic protein G-R1 beads and incubated for 1 h. The supernatant was removed, and beads set apart using a magnetic rack and washed twice with 0.01M PBS 0.03% CHAPS. 100 μL of 0.01M PBS 0.03% CHAPS was added to the beads then the beads and solution were transferred to a new tube in order to reduce any non-specific interaction. The supernatant was then removed, and the beads washed again with 200 μL 0.01M PBS 0.03% CHAPS then 100 μL 0.01M PBS. Elution was performed using 50 μL of 4:1 (v/v) solution of 0.1% FA, then mixed at room temperature for 5 minutes. The elution was collected into new tube and beads were separated from the solution using magnetic rack. A second elution was performed using 100 μL 70% ACN in 0.1% TFA mixed at room temperature for 5 minutes. The second elution was then collected and mixed with the first elution and dried using centrifugation at low pressure.

The dried samples were reconstituted into 20 μL 0.1% FA. The samples were loaded on Evosep tips according to the manufacturer's instructions. The samples were analysed on an Orbitrap Fusion Lumos Tribrid Mass Spectrometer using 88-minute gradient, in data dependant mode in HCD-only mode.

MGF files were generated using MSconvertGUI, from ProteoWizard Version 3.0.18145 using the top 150 most intense ion within each MSMS spectrum. The number of MSMS spectra having simultaneously the 3 fragment ions 580.2937amu 679.3621amu and 780.4098amu are shown in Table 3 under the 4 different conditions described above. Redundancy was removed using a relatively conservative criterion and merging all MSMS found within 0.01 Da of the mass of the precursor.

TABLE 3

| | # MSMS | |
|---|---|---|
| | with | # Redundancy |
| Conditions | y678* | removed** |
| Condition 1 | 21,708 | 6,074 |
| Condition 2 | 26,309 | 6,941 |
| Condition 3 | 27,878 | 7,469 |
| Condition 4 | 25,193 | 5,240 |

*Total number of MSMS events in which the ions 580.2937, 679.3621 and 780.4098 amu are detected
**Conservative redundancy removal (merge anything within 0.01 Da)

The results depicted in Table 3 show that condition 3, which includes IgG enrichment using protein G with standard urea and RP-SPE cleanup, appears to provide the highest level of CDR3 fragment enrichment among the conditions tested.

### Example 13: Enrichment of CDRH3 Fragments Using Cysteine Conversion into Thiol Ethylamine

The rationale of these experiments are as follows: Cysteine side chain are converted into a thiol-ethylamine which allows trypsin (and to a certain extent other lysine endoproteases such as LysC) to cut under those circumstances at the C-terminal side of the modified cysteine residue. By combining cysteine conversion into the lysine analogue thiol-ethylamine, and using trypsin or LysC digestion followed by immune-enrichment against the JC human region, long peptide starting from the end of framework 3 from the heavy chain (cut at the C terminal end of cysteine), and at the opposite a lysine is found at the N-terminal side of the C region (i.e. . . . ASTK/GPS . . . ), therefore it can be possible in some cases to enrich for the full length CDR3. Combination with a second protease after CDR3 enrichment may also allow for a better sequence coverage.

The same plasma sample as that used in Example 12 above was used for this experiment.

A recombinant form of the R1 antibody was used to perform the immunoprecipitation. 250 μg of the recombinant antibody R1 was coupled with 70 μL magnetic protein G slurry (Magne® protein G beads Promega). Initially the 70 μL of protein G slurry was washed twice with 500 μL 0.01M PBS 0.03% CHAPS. The solution antibody R1 and protein G beads was diluted up to 100 μL using 0.01M PBS 0.03% CHAPS then gently mixed at room temperature for 1 hour. The supernatant was removed, and the magnetic beads were washed three times with 500 μL 0.01M PBS 0.03% CHAPS to remove any unbound R1 antibody. A stock solution of antibody R1 coupled to protein G beads was then made in 1 mL 0.01M PBS 0.03% CHAPS.

Beads Digest (IgG Digest on Protein G Magnetic Beads)

40 μL of magnetic protein G slurry was washed twice with 500 μL 0.01M PBS 0.03% CHAPS. 20 μL of plasma was added, and the mixture was diluted up to 100 μL using 80 μL 0.01M PBS 0.03% CHAPS and mixed at room temperature for 1 hour. The supernatant was removed from the protein G-IgG magnetic beads, which were washed twice with 100 μL 0.01M PBS 0.03% CHAPS and once with 100 μL 0.01M PBS and removed supernatant. The concentration of IgG in the plasma was found to be 4.75 μg/μL, thus 20 μL of plasma is roughly equal to 100 μg of IgG. 3 aliquots of 100 μg IgG were prepared.

After a 1-hour incubation at room temperature, supernatant was removed from beads using a magnetic rack and the beads were washed twice with 100 μL 0.01M PBS 0.03% CHAPS followed by 100 μL PBS. 50 μL of water was added as well as 1.5 μL 1M DTT for a final DTT concentration of 30 mM, then incubated at 95° C. for 15 minutes, the supernatant that contains the IgG fraction was then removed from the magnetic protein G beads and transferred to a new tube.

40 μL of 0.5M of 2-Bromoethylamine hydrobromide, BEA solution (BEA powder from Sigma) was made fresh in 0.1M Tris and added to each sample as well as 10 μL of 1M Tris buffer. The samples were incubated at room temperature for 4 h. 10 μL of Tris 1M was added every hour in order to maintain pH around neutral condition.

35 μL of 100% (w/v) trichloroacetic acid (TCA, Sigma) was added to the beads samples to achieve final TCA concentration around 20% then the precipitation was left at 4° C. overnight. The solutions were centrifuged at 23,000×g for 30 minutes and the supernatant was gently discarded without disturbing the pellet. The pellet was washed with 500 μL acetone twice by centrifuging at 23,000×g for 10 minutes then the supernatant was discarded without disturbing the pellet.

The pellet was reconstituted in 4 μL 4M urea, then incubated at 37° C. and mixed for 10 minutes to fully resuspend the pellet. The solution was diluted up to 20 μL using HPLC grade water then 30 μL of 50 mM ammonium bicarbonate was added. 3 different digests were performed: trypsin, LysC and LysC followed by pepsin. 1 μg of enzyme (trypsin or LysC) was used for each digest, then incubated overnight at 37° C. The following day, samples were dried down using centrifugation under low pressure. The pepsin digestion will be described later as it was performed on the peptide elution following immunoprecipitation.

Solution Digest (IgG Enrichment from Plasma Using Agarose Protein G)

6 mL of agarose protein G slurry (3 mL settled Agarose beads, Genscript Protein G resin cat no L00209) was added to an empty 20 mL Biorad® column (Econo-Pac® Chromatography Column, #7321010 polypropylene columns 20 mL bed volume). The Protein G column was conditioned by passing four times 15 mL 0.01M PBS. 4 mL of plasma was centrifuged at 23,000×g for 10 minutes at 4° C. To this 4 mL of plasma, 8 mL of 0.01M PBS was added then filtered using Millex® low protein bind filter (0.45 um PVDF HV cat no SLHVRO4NL Millipore®) using a syringe. The filtered plasma was loaded and passed over the protein G column three times. The protein G column was washed three times with 10 mL 0.01M PBS. IgG fraction was eluted by adding 12.5 mL 0.1M glycine buffer pH 2.7 to beads, the elution was collected in a 15 mL Falcon tube containing 2.5 mL 1M tris to neutralise the glycine elution. The IgG solution was concentrated using a Amicon® Ultra—4, 30 kDa molecular cut-off (cat UFC803024), the solution was centrifuged at 2400×g with 10-minute intervals allowing top up of the IgG solution to concentrate, then the buffer was exchanged using 0.01M PBS. The final concentration of IgG was estimated to be 17 mg/mL with a final volume of 1.1 mL (for 4×1 ml of plasma).

3×100 μg was aliquoted from stock solution of IgG purified from plasma. 0.9 μL of a solution of 1M DTT was added then diluted up to 30 μL using HPLC grade water, for a final DTT concentration of 30 mM. The samples were incubated at 95° C. for 15 minutes.

40 μL 0.5M of 2-Bromoethylamine hydrobromide, BEA solution (Sigma) was made fresh in 0.1M tris and added to each sample as well as 10 μL 1M Tris buffer. The samples were incubated at room temperature for 4 h, with addition of 10 μL 1M Tris buffer every hour in order to maintain pH around neutral condition.

30 μL of 100% (w/v) trichloroacetic acid (TCA, Sigma) was added to the beads samples to achieve final TCA concentration around 20% then left in fridge overnight. TCA precipitation was performed in order to remove excess of BEA/tris solution. The solution was centrifuged at 23,000×g for 30 minutes and the supernatant was removed without disturbing the pellet. The pellet was washed with 500 μL acetone twice by centrifuging at 23,000×g for 10 minutes then the supernatant was gently removed without disturbing pellet.

The pellet was reconstituted into 4 μL 4M urea, then incubated at 37° C. and mixed for 10 minutes to fully resuspend pellet. The suspension was diluted up to 20 μL using HPLC grade water then 30 μL of 50 mM ammonium bicarbonate was added. 3 different digests were then performed as mentioned above, trypsin, LysC and LysC followed by pepsin. 1 μg of enzyme (trypsin or LysC) was added to each digest, then incubated overnight at 37° C. The following day, the samples were dried down under low pressure centrifugation.

Immunoprecipitation

1 trypsin sample and 2 LysC digestions samples from both on-bead preparation and in-solution preparation were reconstituted into 100 μL of 0.01M PBS 0.03% CHAPS then 1 μL of 100 mM PMSF inhibitor was added for final inhibitor concentration of 1 mM.

50 μl of the stock solution of magnetic beads protein G-R1 antibody was used per immunoprecipitation. The supernatant was first removed. Samples were added to the magnetic protein G-R1 antibody beads and incubated for 1 h. The supernatant was removed using a magnetic rack and washed twice with 0.01M PBS 0.03% CHAPS. 100 μL of 0.01M PBS 0.03% CHAPS was added to the beads then the beads and solution were transferred to a new tube in order to reduce any non-specific interaction. The supernatant was then removed, and the beads were washed again with 200 μL 0.01M PBS 0.03% CHAPS then 100 μL 0.01M PBS. The elution was performed by incubating the beads with 50 μL of 4:1 (v/v) solution of 0.1% FA to acetonitrile at room temperature for 5 minutes. The beads were set aside using a magnetic rack and the eluate was collected into new tube. A second elution was performed using 100 μL 70% ACN in 0.1% TFA mixed at room temperature for 5 minutes. The second elution was then collected and mixed with the first elution and dried using centrifugation under low pressure.

One of the LysC digest immunoprecipitations (on beads digest and in-solution) was reconstituted in 21 μL 0.1% FA then 1.25 μL of 0.04 μg/μL pepsin was added. The digest was performed at 37° C. for 15 minutes then inactivated at 95° C. for 3 minutes.

Mass Spectrometry Analysis

The dried samples were reconstituted into 20 μL 0.1% FA. The samples were loaded on Evosep tips according to manufacture procedure. The samples were analysed on an Orbitrap Fusion Lumos Tribrid Mass Spectrometer using 88-minute gradient in HCD-only mode.

MGF files were generated using MSconvertGUI, from ProteoWizard V3.0.18145 using the top 150 most intense ion fragments in MSMS mode. The number of MSMS having simultaneously the 3 fragment ions 580.2937amu 679.3621amu and 780.4098amu are shown in Table 4 for the 2 different digestion condition (on-beads and in-solution and the 3-digestion condition trypsin, LysC and LysC+pepsin). A conservative filter was applied to remove redundancy by counting how many different CDR3 MSMS found within 0.01 Da of the mass of the precursor.

TABLE 4

| | | # MSMS | |
|---|---|---|---|
| Digestion | | with | # Redundancy |
| condition | Enzyme | y678* | removed** |
| beads | LysC + Pep | 259 | 165 |
| beads | LysC | 794 | 464 |
| beads | Trypsin | 4,809 | 1,976 |
| solution | LysC + Pep | 22,857 | 8,233 |
| solution | LysC | 35,778 | 10,155 |
| solution | Trypsin | 34,406 | 9,291 |

As shown in Table 4, notable differences were seen between the bead and solution digestion, in contrast to the results obtained in Example 12. A plausible cause identified is the additional precipitation step with TCA to remove the excess BEA from the solution which could affect the protein recovery from both methods. Digestion with pepsin is less specific thus shorter peptides are obtained and not necessary with the targeted y678 ions.

Sequencing of the cysteine modification with bromoethylamine followed by LysC digestion “in-solution” digestion and R1 CDR3 enrichment gives the highest number of peptide sequence having the y6 y7 and y8 fragments typical of a CDR3 peptide at 35,778 sequences and up to 10,155 non redundant sequences.

An example of a sequence is shown here: the peptide “ARDLGTLMDYWGQGTLVTVSSASTK” (SEQ ID NO:134), the “AR . . . ” sequence is often found adjacent to the framework 3 region and the J region, the CDR3 been fully sequenced (DLGTLMDY). This peptide was found in the 3 in-solution digestions (LysC, LysC-Pep and trypsin); Charge 3+, 891.775839amu (Scan: 40621, Exp. m/z: 891.775839, Charge: 3, PreMH+: 2673.31296, Δ: −0.00589, ppm: −2.20).

Although the present invention has been described hereinabove by way of specific embodiments thereof, it can be modified, without departing from the spirit and nature of the subject invention as defined in the appended claims. In the claims, the word “comprising” is used as an open-ended term, substantially equivalent to the phrase “including, but not limited to”. The singular forms “a”, “an” and “the” include corresponding plural references unless the context clearly dictates otherwise.

### REFERENCES

1. Jackson, K. J. L., Liu, Y., Roskin, K. M., Glanville, J., Hoh, R. A., Seo, K., . . . Boyd, S. D. (2014). Human Responses to Influenza Vaccination Show Seroconversion Signatures and Convergent Antibody Rearrangements. Cell Host & Microbe, 16(1), 105-114. doi:10.1016/j.chom.2014.05.013
2. Jardine, J. G., Kulp, D. W., Havenar-Daughton, C., Sarkar, A., Briney, B., Sok, D., Sesterhenn, F., Ereno-Orbea, J., Kalyuzhniy, O., Deresa, I. et al. (2016) HIV-1 broadly neutralizing antibody precursor B cells revealed by germline-targeting immunogen. Science, 351, 1458-1463.
3. Cheung, W. C., Beausoleil, S. A., Zhang, X., Sato, S., Schieferl, S. M., Wieler, J. S., Beaudet, J. G., Ramenani, R. K., Popova, L., Comb, M. J. et al. (2012) A proteomics approach for the identification and cloning of monoclonal antibodies from serum. Nature biotechnology, 30, 447-452.
4. Bashford-Rogers, R., Bergamaschi, L., McKinney, E., Pombal, D., Mescia, F., Lee, J., Thomas, D., et al. (2019). Analysis of the B cell receptor repertoire in six immune-mediated diseases. Nature, 574 122-126.
5. Chen J, Zheng Q, Hammers C M, Ellebrecht C T, Mukherjee E M, Tang H Y, Lin C, Yuan H, Pan M, Langenhan J, Komorowski L, Siegel D L, Payne A S, Stanley J R. Proteomic Analysis of Pemphigus Autoantibodies Indicates a Larger, More Diverse, and More Dynamic Repertoire than Determined by B Cell Genetics. Cell Rep. 2017 Jan. 3; 18(1):237-247. doi: 10.1016/j.celrep.2016.12.013.
6. Choudhary N, and Wesemann D R, Analyzing immunoglobulin repertoire. Frontiers in Immunology vol 9 article 462 doi.org/10.3389/fimmu.2018.00462
7. Razavi M, Leigh Anderson N, Pope M E, Yip R, Pearson T W. High precision quantification of human plasma proteins using the automated SISCAPA Immuno-MS workflow. N Biotechnol. 2016 Sep. 25; 33(5 Pt A): 494-502. doi: 10.1016/j.nbt.2015.12.008. Epub 2016 Jan. 6.
8. Georgiou G, Ippolito G C, Beausang J, Busse C E, Wardemann H, Quake S R. The promise and challenge of high-throughput sequencing of the antibody repertoire. Nat Biotechnol. 2014 February; 32(2):158-68. doi: 10.1038/nbt.2782. Epub 2014 Jan. 19.
9. Wine Y, Boutz D R, Lavinder J J, Miklos A E, Hughes R A, Hoi K H, Jung S T, Horton A P, Murrin E M, Ellington A D, Marcotte E M, Georgiou G. Molecular deconvolution of the monoclonal antibodies that comprise the polyclonal serum response. Proc Natl Acad Sci USA. 2013 Feb. 19; 110(8):2993-8. doi: 10.1073/pnas.1213737110. Epub 2013 Feb. 4.
10. Xu J L, Davis M M. Diversity in the CDR3 region of V(H) is sufficient for most antibody specificities. Immunity. 2000 July; 13(1):37-45.
11. Guthals A, Gan Y, Murray L, Chen Y, Stinson J, Nakamura G, Lill J R, Sandoval W, Bandeira N. De Novo MS/MS Sequencing of Native Human Antibodies. J Proteome Res. 2017 Jan. 6; 16(1):45-54. doi: 10.1021/acs.jproteome.6b00608. Epub 2016 Nov. 2.
12. Iwamoto N, Hamada A, Shimada T. Antibody drug quantitation in coexistence with anti-drug antibodies on nSMOL bioanalysis. Anal Biochem. 2018 Jan. 1; 540-541:30-37. doi: 10.1016/j.ab.2017.11.002. Epub 2017 Nov. 9.
13. Jason Lavinder, Yariv WINE, Danny Boutz, Edward Marcotte, George Georgiou (2012) PCT/US2012/066454
14. Takashi Shimada, Noriko IWAMOTO (2015) Method for obtaining peptide fragment from antibody by protease decomposition reaction with restricted reaction field Application U.S. Ser. No. 15/556,812
15. Shi B, Ma L, He X, Wang X, Wang P, Zhou L, Yao X. Comparative analysis of human and mouse immunoglobulin variable heavy regions from IMGT/LIGM-DB with IMGT/HighV-QUEST. Theor Biol Med Model. 2014 Jul. 3; 11:30. doi: 10.1186/1742-4682-11-30.

1. A method for obtaining a sample enriched in peptides comprising the third complementarity-determining region of the heavy chain (CDRH3) of an immunoglobulin, the method comprising:
(a) providing an immunoglobulin-comprising sample;
(b) optionally submitting the immunoglobulin-comprising sample to a treatment that modifies lysine residues into residues that are not substrates for lysine endoproteases;
(c) optionally submitting the sample in (a) or (b) to a treatment that modifies cysteine residues into lysine analogue residues or prevents cysteine residues from forming disulfide bonds;
(d) contacting the sample with an endoprotease under conditions suitable for protein digestion to cleave the immunoglobulin into peptides and generate a peptide comprising (i) the CDRH3 and (ii) an epitope comprising the junction (J) region and the first 4 to 25 residues from the constant (C) region of the immunoglobulin;
(d1) optionally inactivating the endoprotease and/or removing from the sample the reagents used for endoprotease digestion;
(e) contacting the peptide-comprising sample in (d) with an anti-CDRH3 peptide antibody or antigen-binding fragment thereof that specifically binds to the epitope, thereby forming complexes of the anti-CDRH3 peptide antibody and the CDRH3 peptides present in the sample; and
(f) dissociating the CDRH3 peptides from the complexes, thereby obtaining a sample enriched in peptides comprising CDRH3 of an immunoglobulin.

2. The method of claim 1, wherein the treatment of step (c) comprises modification of cysteine residues with acrylamide, iodoacetamide or 2-Bromoethylamine hydrobromide.

3. The method of claim 1, wherein the treatment that modifies lysine residues into residues that are not substrates for lysine endoproteases comprises acetylation, dimethylation, guanidization, or carbamylation.

4. The method of claim 1, wherein the immunoglobulin is mammalian immunoglobulin.

5. The method of claim 1, wherein the immunoglobulin is of the IgG class.

6. The method of claim 1, wherein the epitope is located (i) in a region that overlaps the J region and the C region of the heavy chain of the immunoglobulin; or (ii) in the first 15 residues from the C region of the heavy chain of the immunoglobulin.

7. The method of claim 6, wherein the epitope located in a region that overlaps the J region and the C region of the heavy chain of the immunoglobulin is of the sequence VTVSSASTK (SEQ ID NO:1); and the epitope is located in the first 15 residues from the C region of the heavy chain of the immunoglobulin is of the sequence GPSVFPLAP (SEQ ID NO:2), SVFPLA (SEQ ID NO:3) or AST(KMe2)GPSVFP (SEQ ID NO:4).

8-10. (canceled)

11. The method of claim 1, wherein the anti-CDRH3 peptide antibody is a monoclonal antibody comprising the following combination of complementarity-determining regions (CDRs):
VH CDR1: GFSLSSY (SEQ ID NO:5) or a variant thereof having one mutation;
VH CDR2: DANDY (SEQ ID NO:6) or a variant thereof having one mutation;
VH CDR3: YSRDGAIDPYFKI (SEQ ID NO:7) or a variant thereof having one mutation;
VL CDR1: QSSQSVAGNRWAA (SEQ ID NO:8) or a variant thereof having one mutation;
VL CDR2: QASKVTS (SEQ ID NO:9) or a variant thereof having one mutation; and
VL CDR3: AGGYSGEFWA (SEQ ID NO:10) or a variant thereof having one mutation; or
VH CDR1: GFSFSSGY (SEQ ID NO:11) or a variant thereof having one mutation;
VH CDR2: DISGPY (SEQ ID NO:12) or a variant thereof having one mutation;
VH CDR3: TDPTISSSYFNL (SEQ ID NO:13) or a variant thereof having one mutation;
VL CDR1: QSSQSVYKNNRLA (SEQ ID NO:14) or a variant thereof having one mutation;
VL CDR2: LASTLAS (SEQ ID NO:15) or a variant thereof having one mutation; and
VL CDR3: QAYYDGYIWA (SEQ ID NO:16) or a variant thereof having one mutation.

12. The method of claim 1, wherein the anti-CDRH3 peptide antibody is bound to a solid support.

13. The method of claim 12, wherein the solid support are protein A- or protein G-conjugated beads or a monolithic column.

14-15. (canceled)

16. The method of claim 1, wherein the endoprotease is trypsin, a trypsin-like endoprotease, Lys-C, Lys-N, Asp-N, Glu-C, Pro/Ala protease, Sap9, KEX2, IdeS or IdeZ.

17. The method of claim 1, further comprising contacting the sample with a second protease.

18. The method of claim 17, wherein the second protease is pepsin, chymotrypsin, proteinase K, Glu-C or Asp-N.

19. The method of claim 1, further comprising enriching the immunoglobulin-comprising sample in immunoglobulins prior to performing step b, c or d.

20-22. (canceled)

23. The method of claim 1, wherein the immunoglobulin-comprising sample is a biological sample or a cell culture sample.

24-25. (canceled)

26. The method of claim 1, wherein the immunoglobulin-comprising sample is obtained from a subject from an infection, an autoimmune disease, a cancer, or from a vaccinated subject.

27. (canceled)

28. The method of claim 1, further comprising analyzing or characterizing the peptides comprising CDRH3 of an immunoglobulin obtained in step (f).

29-30. (canceled)

31. An anti-CDRH3 peptide antibody or an antigen-binding fragment thereof that specifically binds to an antigen of 5 to 12 amino acids comprising a sequence that (i) overlaps the junction (J) region and the constant (C) region of an immunoglobulin; or (ii) is within the first 15 residues from the C region of an immunoglobulin.

32-34. (canceled)

35. The anti-CDRH3 peptide antibody or an antigen-binding fragment thereof of claim 31, wherein the sequence is VTVSSASTK (SEQ ID NO:1) or GPSVFPLAP (SEQ ID NO:2).

36. The anti-CDRH3 peptide antibody or an antigen-binding fragment thereof of claim 35, wherein the anti-CDRH3 peptide antibody comprises the following combination of complementarity-determining regions (CDRs):
VH CDR1: GFSLSSY (SEQ ID NO:5) or a variant thereof having one mutation;
VH CDR2: DANDY (SEQ ID NO:6) or a variant thereof having one mutation;
VH CDR3: YSRDGAIDPYFKI (SEQ ID NO:7) or a variant thereof having one mutation;
VL CDR1: QSSQSVAGNRWAA (SEQ ID NO:8) or a variant thereof having one mutation;
VL CDR2: QASKVTS (SEQ ID NO:9) or a variant thereof having one mutation; and
VL CDR3: AGGYSGEFWA (SEQ ID NO:10) or a variant thereof having one mutation; or
VH CDR1: GFSFSSGY (SEQ ID NO:11) or a variant thereof having one mutation;
VH CDR2: DISGPY (SEQ ID NO:12) or a variant thereof having one mutation;
VH CDR3: TDPTISSSYFNL (SEQ ID NO:13) or a variant thereof having one mutation;
VL CDR1: QSSQSVYKNNRLA (SEQ ID NO:14) or a variant thereof having one mutation;
VL CDR2: LASTLAS (SEQ ID NO:15) or a variant thereof having one mutation; and
VL CDR3: QAYYDGYIWA (SEQ ID NO:16) or a variant thereof having one mutation.

37-44. (canceled)
